(19) United States
(12) Patent Application Publication
(10) Pub. No.: US 2022/0076851 A1
(43) Pub. Date: Mar. 10, 2022

(54) PATIENT AND SERVICE PROVIDER REMOTE INTERACTION SYSTEM, METHOD AND APPARATUS

(71) Applicant: Nazar Kamangar, Walnut Creek, CA (US)

(72) Inventor: Nazar Kamangar, Walnut Creek, CA (US)

(21) Appl. No.: 17/084,449

(22) Filed: Oct. 29, 2020

Related U.S. Application Data
(60) Provisional application No. 63/076,315, filed on Sep. 9, 2020.

(51) Int. Cl. G16H 80/00; G16H 10/20; G16H 40/20; G16H 10/60

(52) U.S. Cl. CPC G16H 80/00; G16H 10/60; G16H 40/20; G16H 10/20

(57) ABSTRACT

Embodiments of architecture, systems, and methods that enable, e.g., a patient to receive services from a dental service provider and a service provider to interact with and provide services to a patient. Determinations may be made concerning suitability of a service request for remote service, as well as queueing prioritization of a remote service request, based on factors such as: information concerning the service request, patient responses to queries, patient profile information, and patient service history. Content, such as ads or educational information, may be presented to the patient while in queue.

### TECHNICAL FIELD

Various embodiments described herein relate generally to architecture, systems, and methods used to enable client interactions with providers of dental care and other services in-person and remotely.

### BACKGROUND INFORMATION

Traditional approaches for providing dental care services may often provide patients with quality in-person care. However, the convenience and efficiency of service models may be in various ways unsatisfactory for all parties involved, including the services provider, the patient, and where applicable, third party payers (e.g. insurers). For example, patient options to interact with dental service providers are often cumbersome, time-consuming and expensive. In-office appointments may involve scheduling limitations, travel, in-office waiting and substantial expense, coupled for some patients with heightened concerns about potential exposure to infectious disease. Telephonic consultations may be difficult to schedule, with limited ability to communicate issues of concern, cost uncertainty and limited documentation of results. As a result of such issues, individuals often delay in seeking dental care, leading to greater pain and discomfort, as well as more costly and extensive remedial treatments later.

In other circumstances, patients may escalate service demands inappropriately, incurring high costs by seeking emergency treatment from a dentist or even hospital emergency room, when such treatment may not be necessary or could have been avoided by earlier intervention. Patients may have limited ability to identify an appropriate service provider for any given issue, leading to inefficient use of resources.

Meanwhile, information related to oral care and related services is often highly siloed and inefficiently utilized. Patients may have limited visibility into their insurance benefit coverage available for any given services. Patients often have little access to their own records; record requests may be slow, time consuming, and incur substantial costs (whether charged through to the requester or imposed as an administrative burden on the record holder). Patients may have little knowledge or understanding of dental products and services that may be relevant to them. In other situations, patients may be required to provide information multiple times to various service providers and insurers. Limited information availability to insurers may delay payments, cause erroneous coverage determinations, and impose significant burdens on care providers in submitting claims and responding to information requests.

These and other challenges may provide many opportunities for improvement.

### SUMMARY

In accordance with some of the aspects described herein, a platform enables improved interactions between various entities involved in a service provider-client relationship, such as a dental office and a patient, and optionally further with a third party payer such as an insurer or employer.

For example, in accordance with one aspect, a network-connected server facilitates interaction communications between one or more dental service providers and one or more patients, each using a network-connected electronic device, such as (without limitation) a personal computer, smartphone, other mobile communications device, tablet computer, or voice interactive device. A request for remote consultation may be received from a patient electronic device (“PED”). The request may include one or more items of information descriptive of the desired consultation, whether submitted by a patient when initially requesting a consultation or submitted thereafter in response to presenting the patient with questions concerning, e.g., their current condition. The platform them determines the suitability of the request for remote consultation based at least in part upon information provided by the PED, in order to either initiate a service request for a service provider remote session, or direct the patient to seek an alternative service provider (such as an emergency room or other emergency services provider). Information that may be utilized to determine suitability for remote service may include, for example, information provided by a patient to a SPCI server in response to queries for information. Such information may include video or image content (e.g. images captured by a patient's smartphone running a SPCI-app), which video or image content may be analyzed automatically to identify the presence of one or more predetermined conditions that may be factored into a service request suitability determination, such as the presence of blood or the absence of a tooth (particularly wherein the user has prior records accessible to the server that indicate a change in condition, such as the prior presence of the tooth).

If a service request for a service provider remote session is initiated, the patient's request may be placed in queue. In some embodiments, separate queues may be maintained for critical and non-critical services. The patient's service request may be prioritized relative to other service requests based upon a number of prioritization factors, including e.g. evaluation of patient electronic medical records, information provided by the patient regarding the service request, information procured from external systems such as a patient's brushing and other oral hygiene practices, duration of time since submission of the service request, and information available in a patient profile.

Digital content (such as video content, audio content, articles, short textual content, image-based content, or the like) may be presented to users while waiting in queue for remote service, and may be made available to a user on demand via a SPCI-app. Content made available to a patient for playback may include patient educational content, advertising, or other content. Content may be personalized, or selected for presentation from amongst a number of different content items, based on a variety of factors, including information concerning the patient's current service request, past service requests, user profile information, and the like.

Various components may be implemented using machine teaming to, e.g., assist with automatic evaluation of request suitability for remote consultation, and/or to assist with remote service request prioritization (such as for purposes of determining queue priority), and/or for matching of patient consultation requests with available service providers. Feedback may be solicited from service providers and utilized to train such machine learning based algorithms and classifiers.

These and other aspects are described in detail below and in the accompanying drawings.

### BRIEF DESCRIPTION OF THE DRAWINGS

FIG. 1A-C are block diagrams of a service provider-client interactive (SPCI) architectures according to various embodiments.

FIG. 2A is a diagram of communications between a service provider-client interactive (SPCI) client system, a SPCI provider system, a SPCI other providers system, a SPCI server system, and a cloud-blockchain (CBC) system according to various embodiments.

FIG. 2B is a diagram of communications between a service provider-client interactive (SPCI) client system, a SPCI provider system, a payment system, a SPCI server system, and a cloud-blockchain (CBC) system according to various embodiments.

FIG. 2C is a block diagram of SPCI server system communicating data to SPCI provider systems and a SPCI client system according to various embodiments.

FIG. 2D is a block diagram of SPCI server system communicating data to SPCI provider systems and SPCI client systems according to various embodiments.

FIG. 2E is a diagram of a screen of an SPCI provider system according to various embodiments.

FIG. 3A is a block diagram of SPCI server system communicating data to a main screen of an SPCI provider system according to various embodiments.

FIG. 3B is a block diagram of SPCI server system communicating data to a main screen of a SPCI client system according to various embodiments.

FIGS. 3C-3N are diagrams of screens of an SPCI provider system according to various embodiments.

FIGS. 3O-3P and 3R-3AA are diagrams of screens of an SPCI client system according to various embodiments.

FIG. 4 is a block diagram of a SPCI server system according to various embodiments.

FIGS. 5A-5F are flow diagrams illustrating several methods according to various embodiments.

FIG. 6A is a diagram of a first sensor system and neural network architecture according to various embodiments.

FIG. 6B is a diagram of a second sensor system and neural network architecture according to various embodiments.

FIG. 6C is a diagram of a third sensor system and neural network architecture according to various embodiments.

FIG. 6D is a diagram of a data processing module network according to various embodiments.

FIGS. 7A-7C are diagrams of SPCI provider system physical configurations according to various embodiments.

FIGS. 8A-8B are block diagrams of articles according to various embodiments.

FIG. 9 is a flowchart of a process for evaluating and queueing non-critical service requests.

FIG. 10 is a flowchart of a process for evaluating and queueing critical service requests.

### DETAILED DESCRIPTION

In accordance with some embodiments, a service provider-client interaction (SPCI) architecture (400, 420, 100C, 100D, 10) is provided that enables new and/or improved service provider and/or payee interactions with clients during many phases and types of services. In some embodiments, service providers such as dental care providers, medical care providers, and others may provide some or all of their services remotely, in-person, or via combination of remote and in-person interactions with one or more clients. To provide an example only, service providers that provide dental services, and clients (i.e. patients) seeking dental services, are described as one possible application of the SPCI architecture (400, 420, 10).

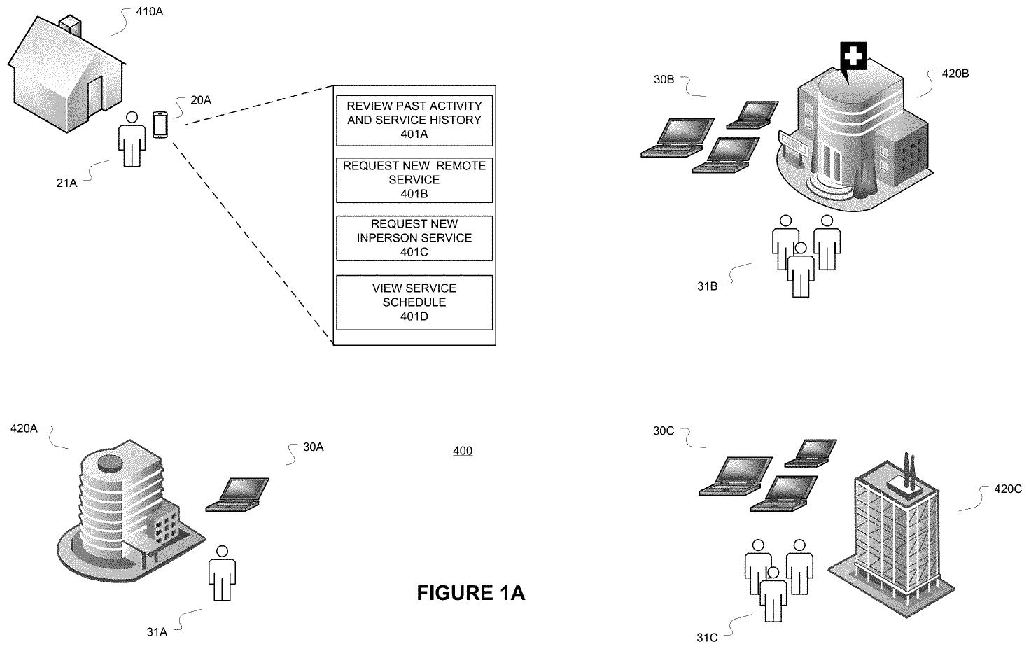

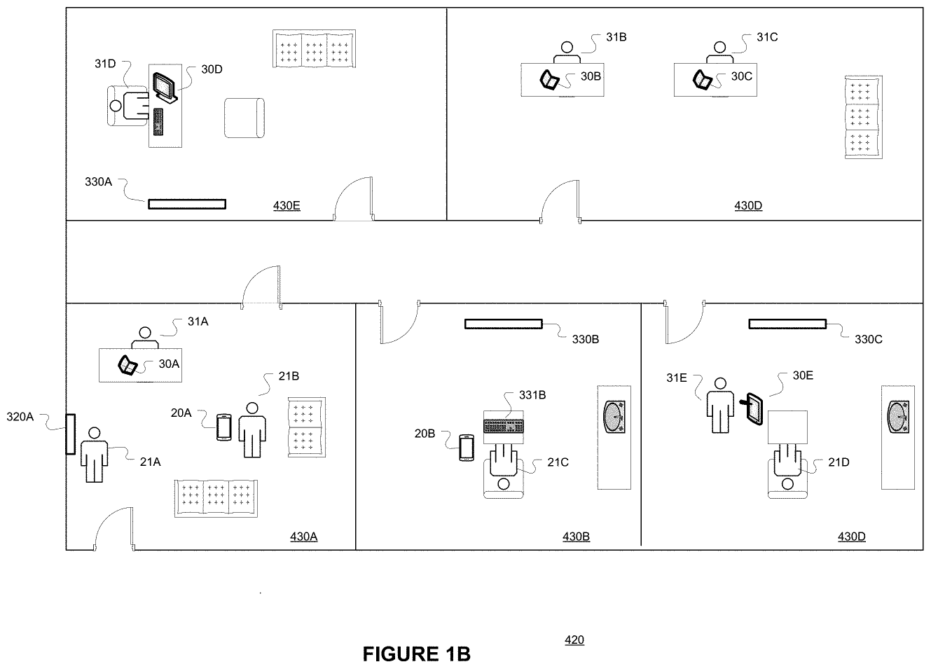

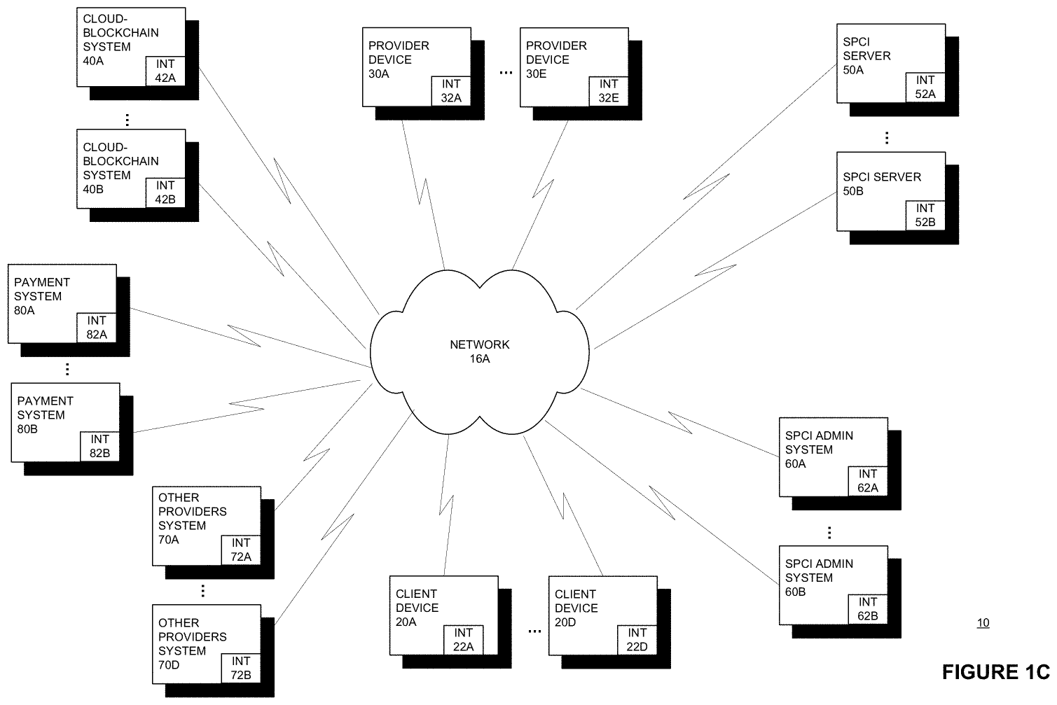

An exemplary environment in which such an embodiment may be implemented is illustrated in FIG. 1A. A client 21A may seek to receive guidance or aid (service) for a dental concern. The client 21A may be located at location 410A, such as the client's home or work location. A dental service provider such as a dentist, dental assistant, support staff, hygienist, or other device service provider may be physically separate from the client 21A, such as provider 31A at location 420A (home or office), providers 31C at a medical office 420C, or providers 31B working at an urgent care or emergency center 420B. A client 21A seeking assistance (service) may use their network-connected electronic device 20A (e.g. a smartphone, other mobile communications device, smart watch, personal computer, or the like) to invoke a SPCI application (such as a mobile app) or webpage that enables them to select one or several support options 401A-401D, as shown in FIG. 1A or described hereinbelow.

### History Request—401A

A client 21A using CED 20A may be provided with immediate, on-demand access to a variety of personal and service records, via a SPCI mobile app or other computer application or webpage (a “SPCI-app”). For example, a client 21A may be able to review past activity and service history uploaded or memorialized by the SPCI architecture 400 (SPCI-history) by accessing selection 401A on the SPCI-app. A client 21A via their CED 20A may be to then view information such as: past chat dialog with a provider 31A-C; prior video consultations with a provider 31A-C (in some embodiments, including date/time/duration information, recordings of sessions, or computer-generated transcripts of sessions); and personal medical or dental records (such as x-rays or other medical/dental imaging, laboratory test results, prescriptions, and the like). In some embodiments, server 50 may query separate electronic medical records (EMR) systems in order to retrieve medical or dental records associated with a given user or patient. Such EMR may subsequently be, e.g., made accessible to a user via a history request 401A, made available to service providers in remote or in-person consultations, utilized as a factor for prioritization of remote service requests, and/or used for automated identification of predetermined conditions, whether conditions exhibited directly within the EMR or conditions determined by comparing EMR content to additional information provided by the patient (e.g. image-based identification of a missing tooth that is present in an EMR x-ray image).

### Remote Service Session Request—401B

For immediate or initial support, aid, or guidance, a client 21A may request a new remote service session (selection 401B). When a client 21A requests a new remote service session selection 401B, architecture 400 may enable a remote session between the client 21A and a service provider 31A-C (providers) via their respective electronic devices 30A-C. As described in more detail elsewhere herein, architecture 400 may direct or schedule a client 21A for a remote or in-person session with a provider 31A-E at locations 420A-C based on information available to the SPCI. Information that may be utilized by the SPCI for session scheduling and prioritization (i.e. “prioritization factors”) includes, inter alia: the client's 21A SPCI-history; one or more questions answered by the client 21A via the SPCI application or web interface (SPCA-app) on CED 20A (SPCI-questions); and information accessed from network-connected third party systems (e.g. patient brushing and other oral hygiene practices as monitored by a smart toothbrush cloud service), other devices (e.g. a Bluetooth-connected smart toothbrush), and/or other data stores or applications (e.g. importation of brushing or other information from other apps or system data stores within CED 20A). Based on such infornation, the SPCI may initiate a remote session (preferably with prioritization triaged based on information available to the SPCI), direct client 21A to in-person session (e.g. visiting an emergency room), or provide client 21A with patient education media or other media content (e.g. providing consultation or advice concerning the patient's condition using pre-recorded content items).

### In-Person Service Session Request—401C

For immediate or initial service, a client 21A may request an in-person service session via selection 401C. In an embodiment, when a client 21A requests an in-person service session 401C, architecture 400 may first enable a remote session between the client 21A and a service provider 31A-C via their devices 20A-D, 30A-E to determine priority, scheduling (if any) and service provider selection for an in-person session. In an embodiment, architecture 400 may also directly schedule a client 21A for an in-person session with a provider 31A-E at locations 420A-C based on SPCI-history and SPCI-questions. Additionally or alternatively, architecture 400 may enable a fully remote session between the client 21A and a provider 31A-C via their respective devices 20A-D, 30A-E based on e.g. SPCI-history and SPCI-questions.

### View Service Session Schedule—401D

To determine or verify sessions (if any) that architecture 400 has scheduled for a client 21A, client 21A may select to view their service session schedule via CED 20A (selection 401D). Architecture 400 may show remote or in-person sessions scheduled for the client 21A. For example, for a remote session request, a client 21A may be in a queue to interact with a service provider 31A-E via their respective service provider electronic device (SPED) 30A-E. A client 21A may also be able to confirm, revise, or cancel a pending remote or in-person service session for a provider 31A-E in an embodiment. SPEDs 31 and CEDs 21 may be any of a variety of network-connected electronic devices facilitating digital communications with their users, such as a personal computer, smartphone or mobile phone, other mobile communications device, tablet computer, smart television, or voice interactive device such as Amazon Alexa. It is contemplated and understood that a given user may utilize multiple different CED, and a given service provider may utilized multiple SPED.

### Exemplary Remote Service Session Architecture

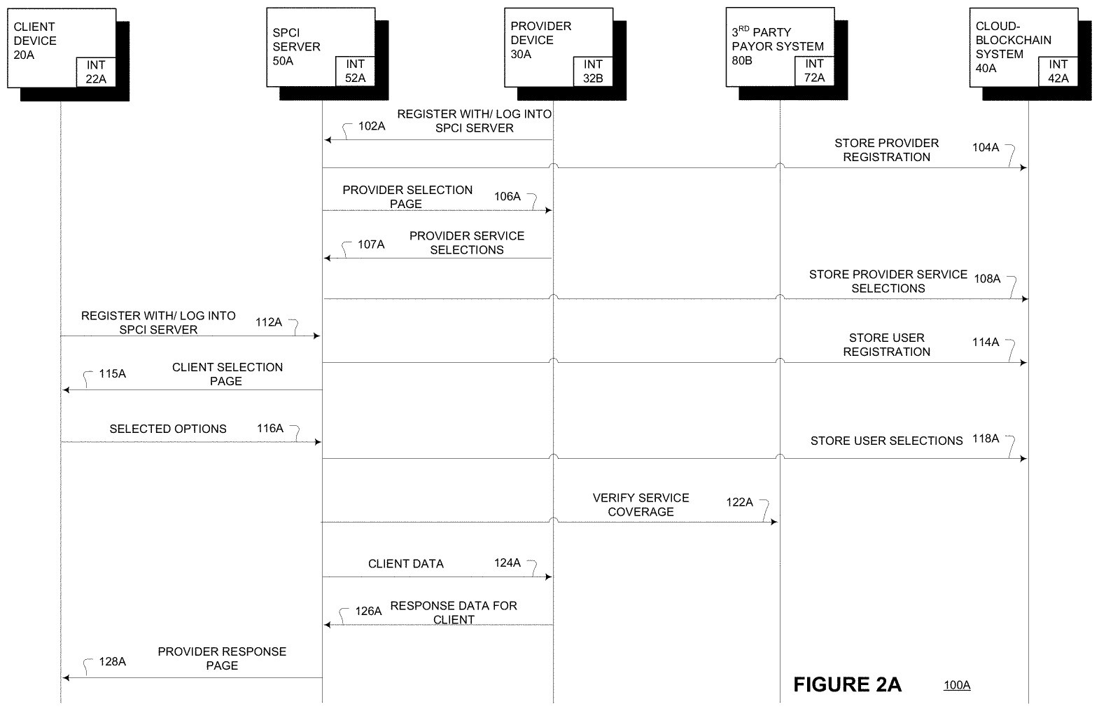

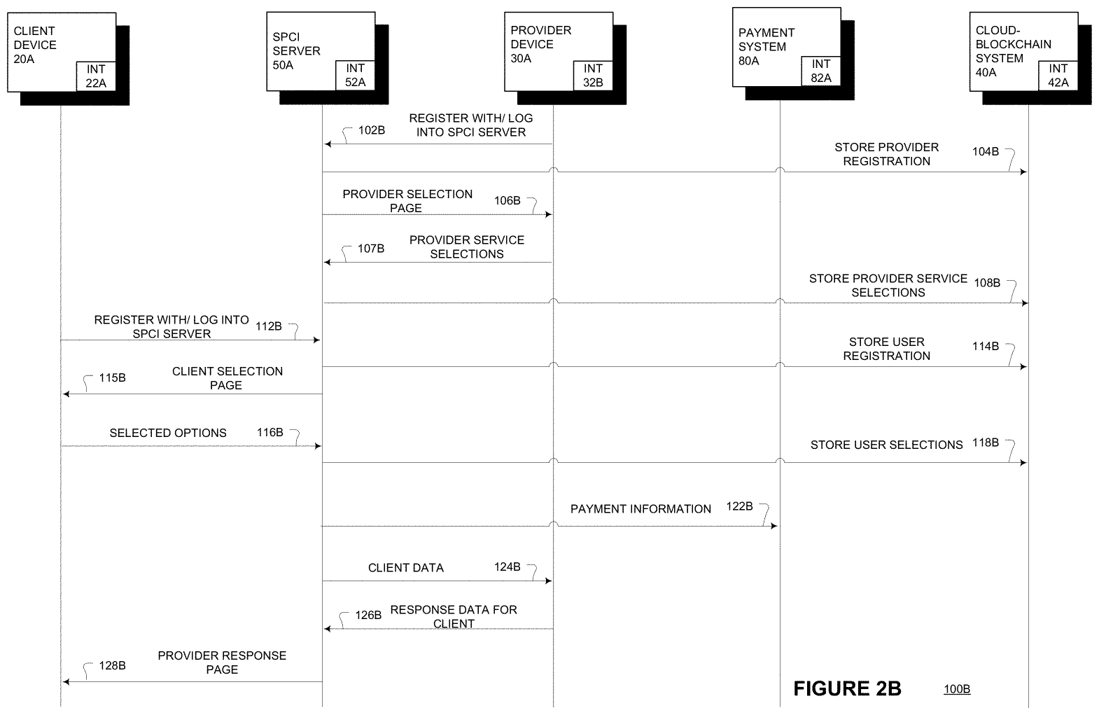

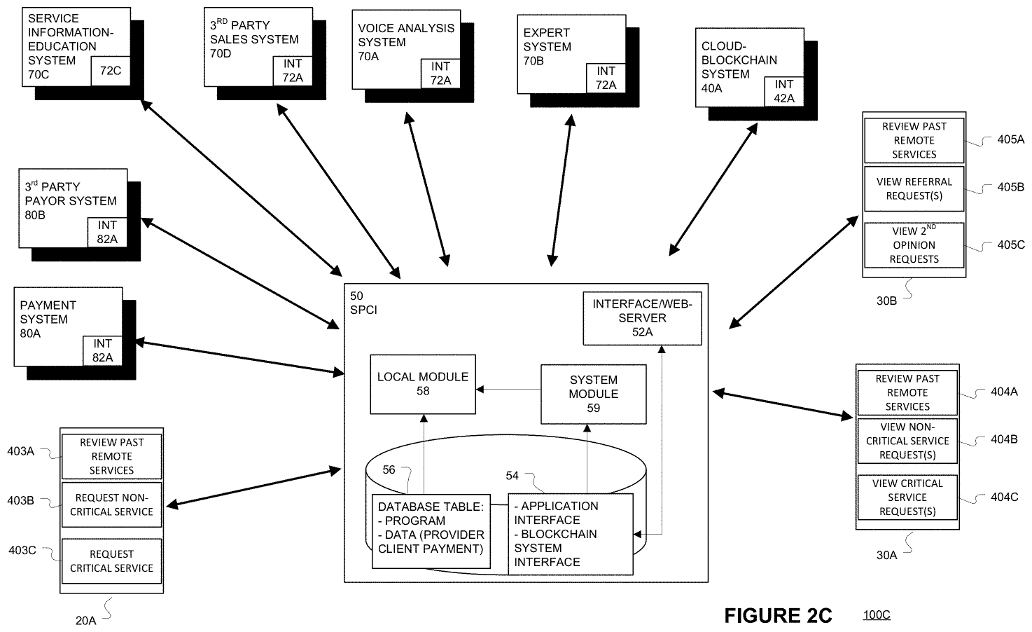

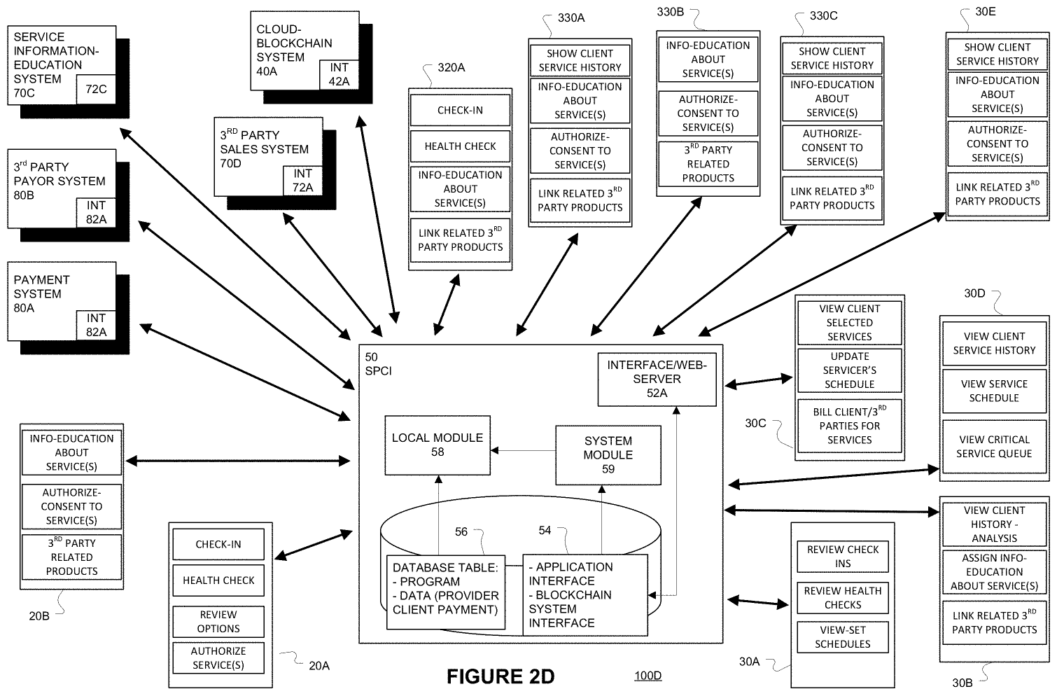

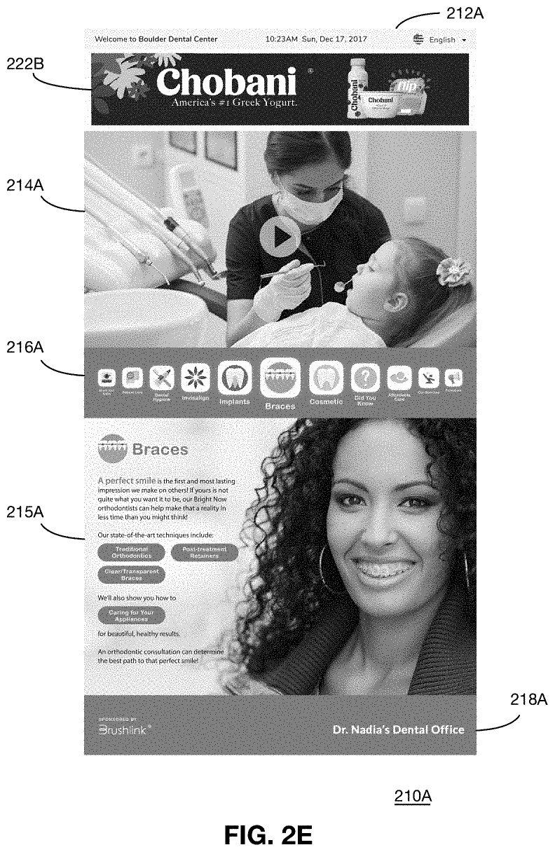

In accordance with some embodiments, the SPCI may be utilized to enable remote service provider consultations, such as tele-dentistry appointments or telehealth consultations, as a service delivery option within an integrated local-remote service platform. FIG. 2C is a block diagram of an architecture 100C that may be employed for SPCI remote session activities in an embodiment. Architecture 100C may include a SPCI server 50 that provides data to SPCI-apps operating on devices 20A, 30A, 30B, such as webpage data for web applications or data for other applications (such as SPCI specific client or provider mobile applications). The SPCI server 50 may also communicate with other providers or services, e.g. via devices 40A, 70A-D, 80A-B to receive, store, and process session related data. The SPCI server 50 may include its own databases 54, 56, modules 58, 59, and interface web-server 52A to process session related data and communicate with devices 20A, 30A, 30B, 40A, 70A-B, 80A-1 in architecture 100C.

When a client 21A via their device 20A requests a remote service session (401B), a SPCI-app on their device 20A may provide several options 403A-C, as illustrated in FIG. 2C. Via the SPCI-app operating on device 20A, a client 21A may be able to review past remote service sessions (403A), including communications between the service providers and SPCI-history in an embodiment; request non-critical service (403B); or request critical service (403C).

Different service options may be provided to service providers, e.g. via provider devices 30A and 30B, including: review past remote services rendered by the provider 404A, 405A; view non-critical service requests 4048 (e.g. requests in queue for the provider); view critical service requests 404C (e.g. requests in queue for the provider), review referral requests 40513, and view second opinion requests 405C.

### Non-Critical Remote Service Session Request

In an embodiment, a client 21A may be able to request a non-critical remote service session (request 403B). In the context of an integrated dental services platform, request 403B may be utilized for patient issues such as requesting consultation on oral hygiene practices, learning about treatment options or available oral care products, or asking questions about non-acute conditions.

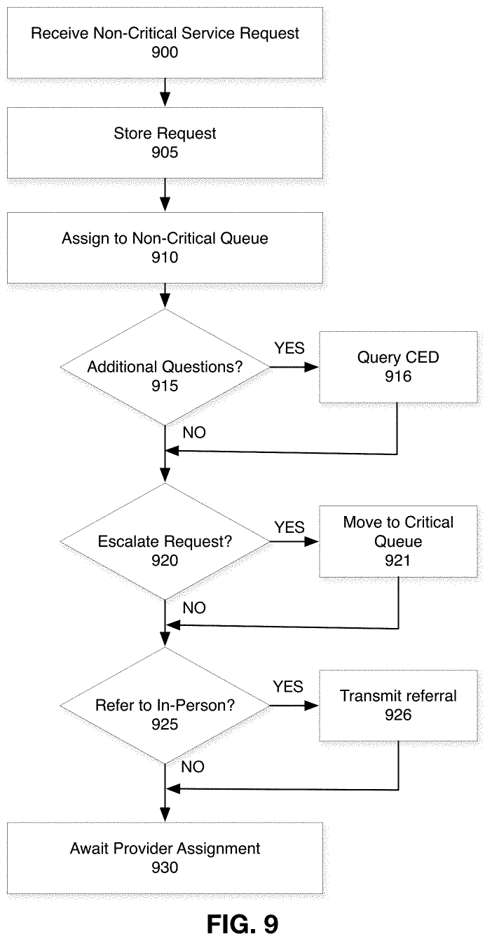

FIG. 9 illustrates an exemplary process for a non-critical remote service session request 403B. In step 900, SPCI server 50 receives a request 4038 from client device 20A. The request 403B may include a status of service wanted, summary, and other data related to the request (such as, in a dental application, a photograph or video showing the portion ofa patient's mouth in question). SPCI server 50 receiving the request 403B may perform a number of functions in an embodiment. In step 905, SPCI server 50 may store some or call data contained within or relating to the request 4038. In some embodiments, request information received from PED 20A may be stored in a secure remote interactive service database, such as networked-based distributed ledger system 40A providing an immutable or nearly immutable record of client requests. In step 910, SPCI server 50 may assign the request to the non-critical service session queue maintained on SPCI server 50 and accessible to a SPCI-app on one or more provider devices such as provider device 30A.

Upon receiving a request, SPCI server 50 may make a determination as to whether the requesting client should be presented with additional questions (i.e. requests for information) (step 915). The determination of step 915 may be made based upon, inter alia, information received by SPCI server 50 in request 403B. If so, SPCI server 50 may query CED 20A for additional information, with the requests for additional information presented to client 21A via the SPCI-app on their CED 20A (step 916). The SPCI server 50 may use an expert system 70B and/or voice analysis system 70A to form and present the additional questions, preferably customized or personalized based on e.g. the content of request 403B and/or related SPCI-history for the requesting user. For example, if a request in step 900 is categorized as a request for oral hygiene consultation and includes a photograph of a patient's teeth, in step 915 SPCI server 50 may evaluate the photograph to identify a broken tooth; and in step 916, client 21A may be further queried (e.g. via questions presented on CED 21A via SPCI-app) as to whether the patient's tooth has been newly broken in the preceding week.

In step 920, SPCI server 50 determines whether the non-critical request 403B should be escalated to a critical request queue. This determination may be made, in some embodiments, based upon information provided by client 21A in the original request (step 900) and/or in response to follow-up questions (step 916). In some embodiments, other sources of information may also be used, such as information accessed from network-connected third party systems (e.g. patient brushing and other oral hygiene practices as monitored by a smart toothbrush cloud service), other devices (e.g. a Bluetooth-connected smart toothbrush), and/or other data stores or applications (e.g. importation of brushing or other information from other apps or system data stores within CED 20A). Ifa determination is made to escalate, the client's request is moved to a critical service request queue (step 921). For example, in some embodiments, if a patient indicates that a tooth has been recently broken as a result of trauma, it may be desirable to escalate the patient's request to a critical queue. In some embodiments, the critical queue may be prioritized more highly for rapid handling, and/or assigned to different service providers (such as service providers having a higher level of expertise and/or training relevant to responding to critical issues).

While the embodiment of FIG. 9 contemplates maintaining separate queues for critical and non-critical requests, it is contemplated and understood that in other embodiments, a single request queue may be maintained, and escalation of a request to a “critical queue” may refer to escalation of a priority level or categorization of the request, thereby impacting the order in which the request is handled relative to other requests. In yet other embodiments, prioritization may be based on a variety of prioritization factors, such as information provided in request 403B, information responsive to questions in step 916, prior patient history, the length of time that the patient has already waited for service, application of machine learning-based prioritization algorithms, expert system-based prioritization scoring, or other factors and systems.

In some embodiments, SPCI server 50 may also utilize information provided in connection with a request to determine whether the requesting client should be referred directly to an in-person service provider (step 925). This may be desirable, for example, where information presented in the original request or in response to follow-up questions suggests that the nature of the issue of such a nature that it cannot be effectively addressed by remote consultation, or is of a severity level that immediate in-person attention is required (such as by an urgent care or emergency facility 4201). If so, in step 926, SPCI server 50 transmits an in-person referral recommendation to client 21A via PED 20A (step 926). Such a referral may include, for example, a name and address of recommended service provider, contact information to schedule an appointment, and/or an automatically-scheduled appointment time.

As referenced elsewhere herein, machine learning techniques may be beneficially utilized in connection with a variety of aspects of the SPCI. For example, machine learning techniques may be utilized to optimize the automated determination of suitability of a request for remote consultation for a remote session, as opposed to an in-person service consultation. Such a machine learning component may be implemented internally by server 50, externally (e.g. via a RESTful API integrating to a third party and/or separately hosted machine learning pipeline), or otherwise. In the event that a remote consultation is initiated, server 50 may solicit feedback as to whether the patient's need was well-suited for remote consultation, and that feedback may be utilized as ongoing training input to the machine learning component. Similarly, a machine learning component (whether implemented by server 50, externally, or combinations thereof) may be utilized as a priority classifier to prioritize patients' requests for remote service consultations in queue; and feedback may be requested from service providers having conducted a remote consultation as to the priority level the service provider would have assigned to the consultation request, which response may be utilized as training feedback for the machine learning priority classifier.

Various criteria may also be considered in assigning remote service requests to a subset of potential service providers, including: a specialization with which the remote service provider is associated, as compared the details of the service request; the availability of remote service providers (whether scheduled or reported by the service provider in real time), and records of prior interactions between a patient and service provider (e.g. prioritizing service providers having previously treated the patient or for whom a prior interaction was rated highly). In some embodiments, machine learning components or expert system components may also be utilized to assist in matching of service requests with service providers; inputs may include information from the patient concerning the consultation request, information from the patient profile or past services; provider specializations; and other information. Service providers may provide feedback as to the appropriateness of the patient match, and such feedback may be utilized as training input for the matching algorithm.

If the request is maintained in a queue for remote service, client 21A may then await connection with a provider for communication between CED 20A and a provider electronic device (step 930).

### Targeted or Personalized Content Delivery On Queue

In some embodiments, clients awaiting providers may be presented with content via their SPCI-app while waiting. Content delivered while waiting on queue may include, for example, advertising or patient education information. Furthermore, content delivered on queue may be highly targeted or personalized, based on detailed information concerning client 21A maintained by or accessible to SPCI server 50. While examples are described herein with reference to a remote dental services application, it is contemplated and understood that the systems and methods described may be equally applicable to telehealth consultations or other remote services.

For example, information received by SPCI server 50 in connection with a service request (e.g. in step 900) and/or in response to further query (e.g. in step 916) and/or associated with the requesting client in a user profile maintained by SPCI server 50, may be utilized to target or personalize content delivery. In some embodiments, content may be selected for playback to a given user while waiting in queue, that is deemed likely to be of interest to the user, whether the delivered content comprises advertising, patient education materials or other content. For example, in some embodiments, if a user initiates a non-critical service request for consultation on oral hygiene and the user's historical service information indicates recent orthodontal work, during step 930 the user may be presented with (a) advertising for orthodontal appliance cleaning products; and/or (b) instructional videos on cleaning of orthodontal appliances.

In some embodiments, content sponsorship may also be utilized (exclusively or as one factor in a recommendation or personalization algorithm) in selecting content for delivery to user 21A while waiting on queue. For example, toothpaste manufacturers may bid for advertising delivery to targeted user cohorts (e.g. users seeking consultation about dental hygiene practices). Because SPCI server 50 has access to a particularly rich and varied ecosystem of information relevant to not only a user 21A, but also the user's contemporaneous interests (e.g. as expressed via current and/or historical requests for service or other prior transactions), SPCI server 50 may be particularly effective in selecting and delivering content that is timely and relevant to user 21A.

In addition to transmitting content such as patient educational content and/or targeted advertising while a patient is in queue for a remote consultation, the SPCI platform may also deliver targeted content (whether patient educational, advertising, or both) via a user 21A's mobile app, when the patient's device 20A is determined to be located within a dental service provider's offices. Thus, content relevant to a patient's current condition can be delivered to the patient while waiting to visit with a dental professional, thereby engaging the patient and potentially reducing patient dissatisfaction associated with any waiting period.

### Critical Remote Service Session Request

In an embodiment, a client 21A may be also able to request a critical remote service session (request 403C). In the context of an integrated dental services platform, request 403C may be utilized for patient issues such as acute pain, a newly broken tooth, broken orthodonture, or the like.

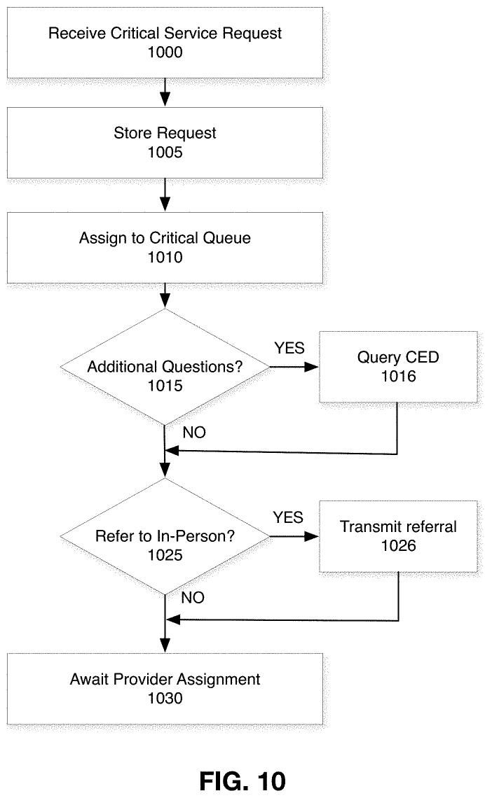

FIG. 10 illustrates an exemplary process for a critical remote service session request 403C. In step 1000, SPCI server 50 receives a request 403C from client device 20A. The request 403C may include a status of service desired, summary, and other data related to the request (such as, in a dental application, a photograph or video showing the portion ofa patient's mouth in question). A SPCI server 50 receiving the request 403C may perform a number of functions in an embodiment. In step 1005, the SPCI server 50 may store some or all data contained within or relating to the request 403C. In some embodiments, request information received from CED 20A and other transactions in which SPCI server 50 engages may be stored in a secure remote interactive service database, such as a network-based distributed ledger system 40A (which may be a blockchain), providing an immutable or nearly immutable record of client requests and optionally other transactions in which SPCI server 50 engages. In step 1010, SPCI server 50 may assign the request to a critical service session queue maintained by SPCI server 50 and accessible to SPCI-app on one or more provider devices such as provider device 30A.

As with the non-critical queue workflow, upon receiving a request, SPCI server 50 may make a determination as to whether the requesting client should be presented with additional questions (step 1015), and if so, additional questions may be presented to user in step 1016 (similarly to step 916). Optionally, a determination may be made as to whether critical queue requests should be de-escalated to the non-critical queue (i.e. the inverse of steps 920 and 921), although it may be desirable not to do so to avoid erroneously de-escalating a genuinely critical patient request.

As with the non-critical request workflow of FIG. 9, SPCI server 50 may also utilize information provided in connection with a critical service request to determine whether the requesting client should be referred directly to an in-person service provider (step 1025). If so, in step 1026, SPCI server 50 transmits an in-person referral recommendation to client 21A via PED 20A (step 1026). Otherwise, if the request is maintained for remote service, client 21A may then await connection with a provider for communication between CED 20A and a provider electronic device (step 1030).

### Payment Processing

In services areas such as dental, payer responsibility may be complex and may vary by service or product. For example, some types of services may be partly or wholly paid by an insurer or a self-insured employer. Some services may entail full or partial payment by a patient, either directly or via a Health Savings Account or Flex Spending Account. Some services may be paid for via a subscription service. Similarly, oral care products may be payable by, iner alia, some combination of an insurer, employer, patient out-of-pocket, patient HSA or patient FSA. Thus, in some embodiments, it may be highly desirable to implement automated payment and billing functionality, as described herein throughout, thus potentially providing additional clarity to patients, and potentially improved collection time and reduced administrative burden for providers and payers.

In some embodiments, the SPCI may facilitate online direct payment by a client 21A, e.g. via the SPCI-app on their device 20A, to pay for a requested or completed remote service session or other product or service. The SPCI-app may enable a client 21A to pay via a 3^{rd} party (such as insurance company) when applicable or other electronic payments such as Paypal®, credit card, EFT, bank checking account, Venmo®. The SPCI server 50 may communicate insurance information required and received from a SPCI-app on a device 20A to a 3^{rd} party payor system 80B for processing, confirmation, and payment. The SPCI server 50 may also communicate electronic payment information received from a SPCI-app on a device 20A to a payment system 80A for processing, confirmation, and payment. The SPCI server 50 may forward payment from system 80A or 80B to a service provider 31A, B based on their election as applicable in an embodiment.

### Client Education

In some embodiments, the SPCI server 50 and/or device 20A implementing an SPCI-app, may enable presentation of informational content to a user 21A, such as educational information about various services relating to a client's service request. Such content may be presented automatically by SPCI server 50, automatically by operating of device 20A implementing SPCI-app, at the request of a service provider 31A, 31B, and/or upon request of a client 21A (e.g. via searching for content using device 20A and the SPCI-app, in some circumstances interacting with search module 88A within server 50). Information received by SPCI server 50 relating to a user's service needs or condition (e.g. information received in steps 900, 916, 1000 or 1016, information stored in distributed ledger 40A, and/or information stored within a database implemented by server 50A) may be utilized to personalize and/or prioritize selection of client education content for presentation to client 21A.

The content to be presented to client 21A may include, inter alia, articles, multimedia such as videos, webpages, audio and other data. The content may have subject matter relating to a current service request from client 21A, a past service request from client 21A, information deemed relevant based on profile or biographical information associated with client 21A, or other subject matter. Video may be provided via a third party online video hosting source such as YouTube®, Facebook®, Vimeo, and others. Other content may likewise be hosted externally.

In some embodiments, the SPCI server 50 may receive service-related educational information from an education system 70C, which may be hosted by another service provider. The education information may include information about the requested service(s) and related services in an embodiment.

In some embodiments, client education information may be presented via a SPCI-app while a patient is waiting in queue for a remote service session. In some embodiments, client education information may be presented via in-app content, made available for a web portal, and/or transmitted to a user via messaging (such as email, SMS, or in-app notifications from the SPCI-app).

### Related Services-Products Sales

In an embodiment, the SPCI server 50 automatically or at the request of a service provider 31A, 31B may enable a client 21A via their SPCI-app (on their device 20A) to purchase products or services related to their service request. For example, for a dental service, the related products may be dental products to prevent future service (e.g. toothpaste, fluoride rinse, a power toothbrush, a brushing coaching aid, or the like) or to help address any current dental issues (e.g. pain relievers, whitening products, or the like). In an embodiment, the SPCI server 50 may provide related products or services information or links for their purchase from a 3^{rd} party sales system 70D, which may be hosted by another service provider. The SPCI server 50 may also enable the SPCI-app of a device 20A to communicate directly with a 3^{rd} party sales system 70D to purchase related products or services.

### Integrated In-Person Service Session Environment

In addition to enabling remote consultation sessions and/or remote access to information, the SPCI platform may also provide functionality when a user is physically present at a service provider's location. Thus, the SPCI platform and SPCI-app may provide a wholly-integrated platform for monitoring, managing and participating in all aspects of a user's services, whether remote or in-person. In particular, a user's SPCI-app implemented on a user's personal electronic device (e.g. device 20A, 20B) may provide different functionality based on whether the user is present in a service provider's in-office setting, or remotely located.

For example, SPCI architecture 400 may be employed at a service providers 31A-E location 420 during the in-person service session for one or more clients. As shown in FIG. 1B, a service provider location 420 may include a number of rooms 430A-E that serve different functions (e.g. examination rooms, operatories, reception areas, or consultation offices). In an embodiment, one or more rooms 430A-E may include service provider devices 30A-E, 320A, and 330A-C that may be running an SPCI application or displaying a SPCI webpage (SPCI-app). The service provider SPCI-app may be used by a service provider 31A-E to prepare or conduct an in-person session for a client. An SPCI-app may also be used by a client 21A on a service provider device 30A-E, 320A, and 330A-C or their device 20A-B to conduct-perform various sessions activities.

In some embodiments, a service-provide SPCI-app may be utilized for various aspects of patient check in within an in-person service environment, including a patient health screening. Reception area room 430A may include a service provider device 320A located therein. Device 320A may be a computing device kiosk, such as a floor-standing kiosk or a wall-mounted kiosk. Patients entering room 430A may be directed to check in using kiosk device 320A.

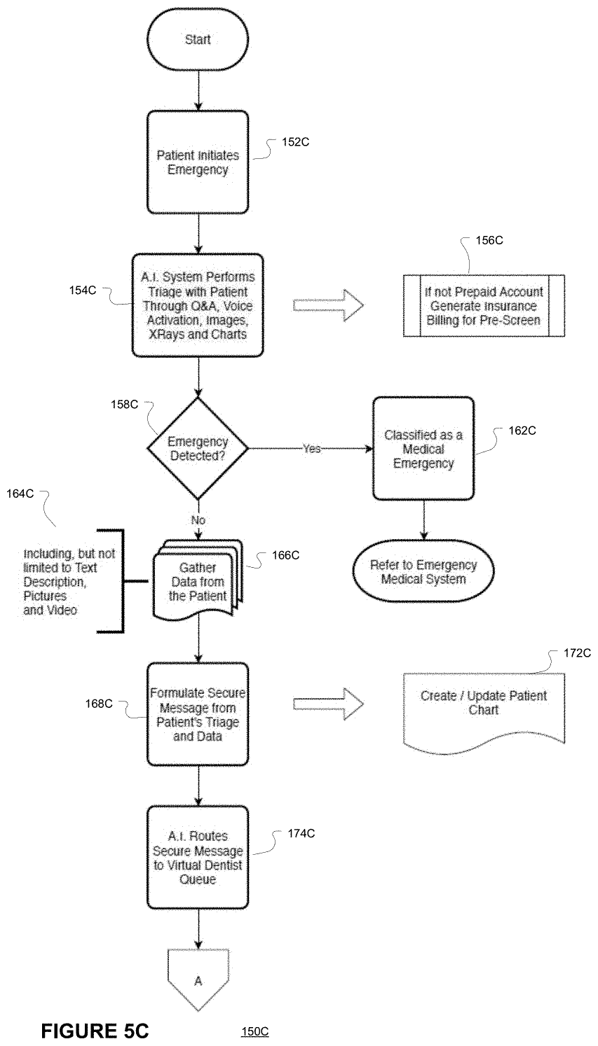

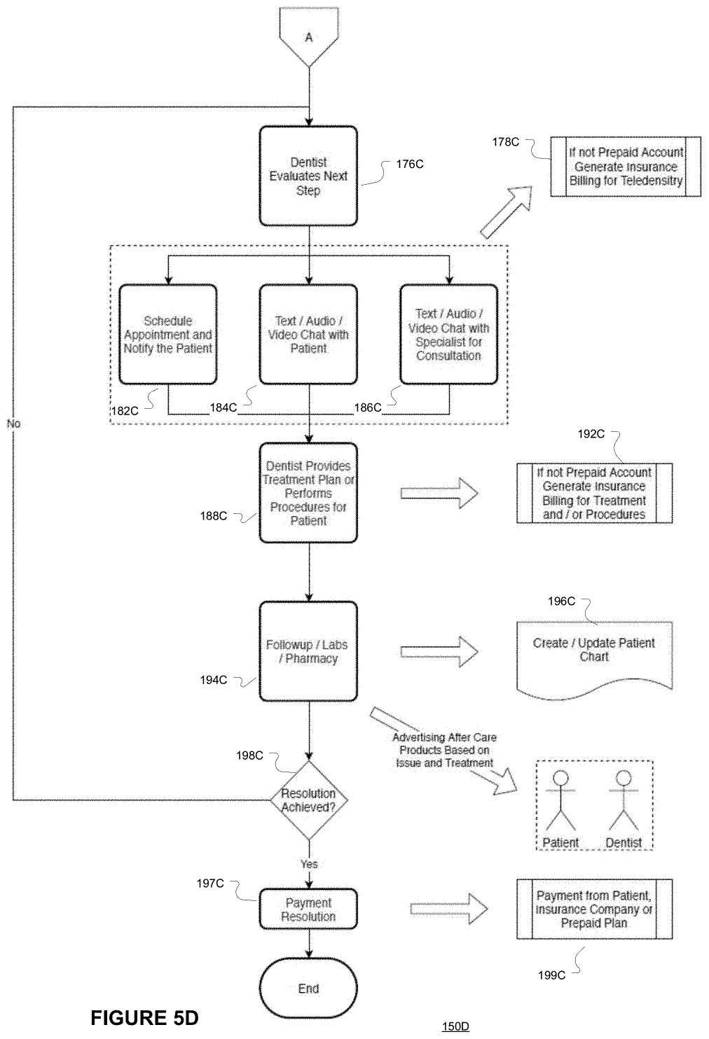

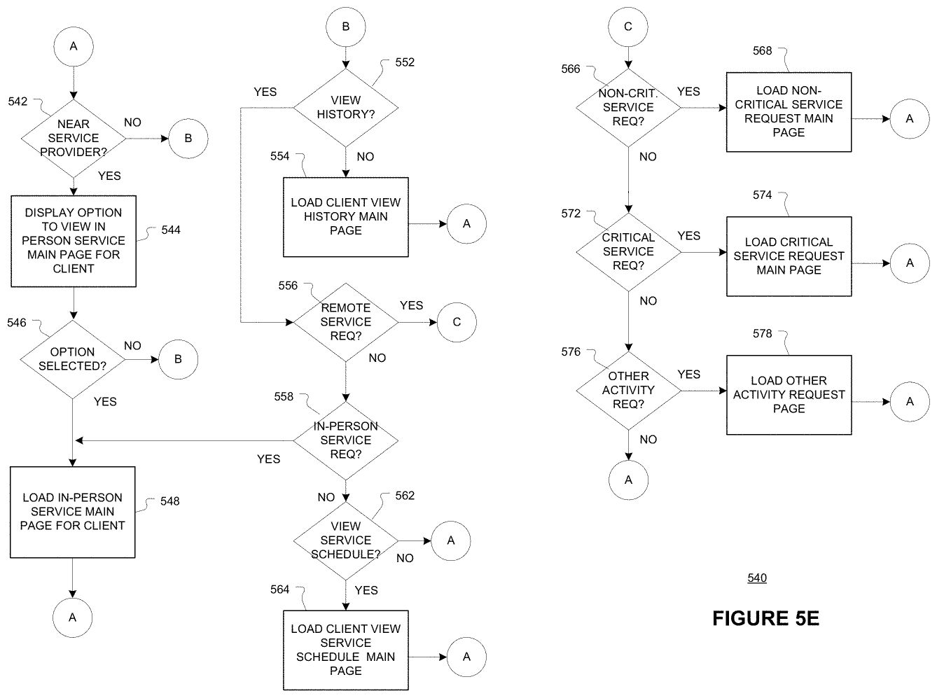

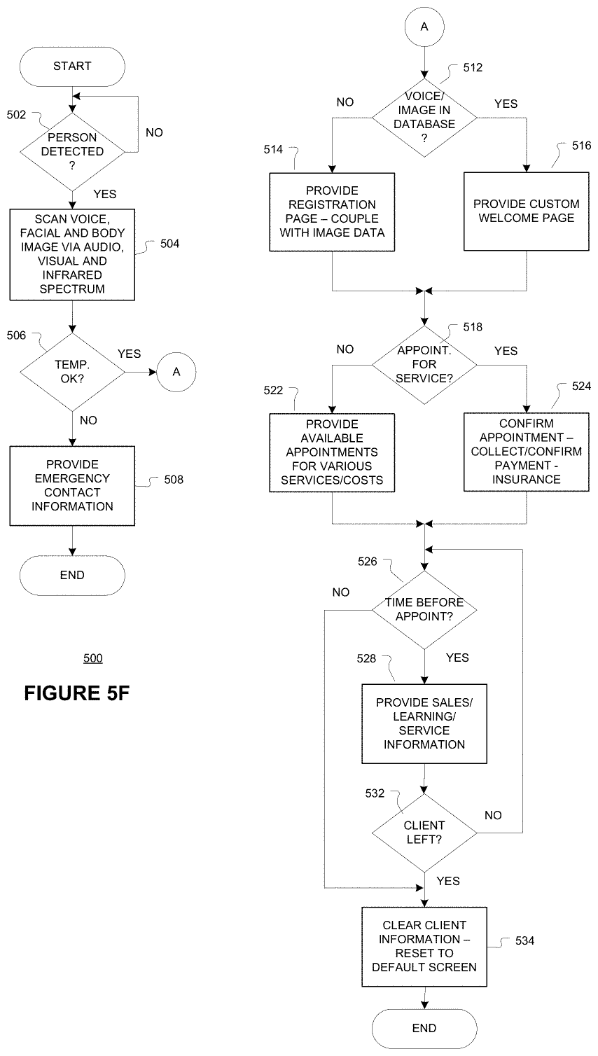

A service provider SPCI-app of a device 320A may automatically detect when a person-client is near the device 320A (activity 502 of algorithm 500 of FIG. 5F). Then the SPCI-app of a device 320A may scan the voice, face, body image of the detected person using different frequency spectrums including audio, visual, and infrared spectrum (activity 504).

In some embodiments, service provider device 320A may be utilized to implement a health screening, before checking a patient in for an in-person service appointment. For example, device 320A may include an infrared scanner configured to perform an infrared scan of a client standing before device 320A to determine their current temperature based on measurement of infrared radiation, and confirm that the person's temperature is within acceptable limits or direct the client-person to an emergency, urgent care, or similar facility 420A if their temperature is elevated beyond current acceptable limits (such as during an outbreak in the community) (activities 506, 508). The health screening performed by device 320A may additionally include querying the user with questions concerning their health condition, such as presence of body aches, cough, other current condition, or recent exposure to potentially infectious individuals.

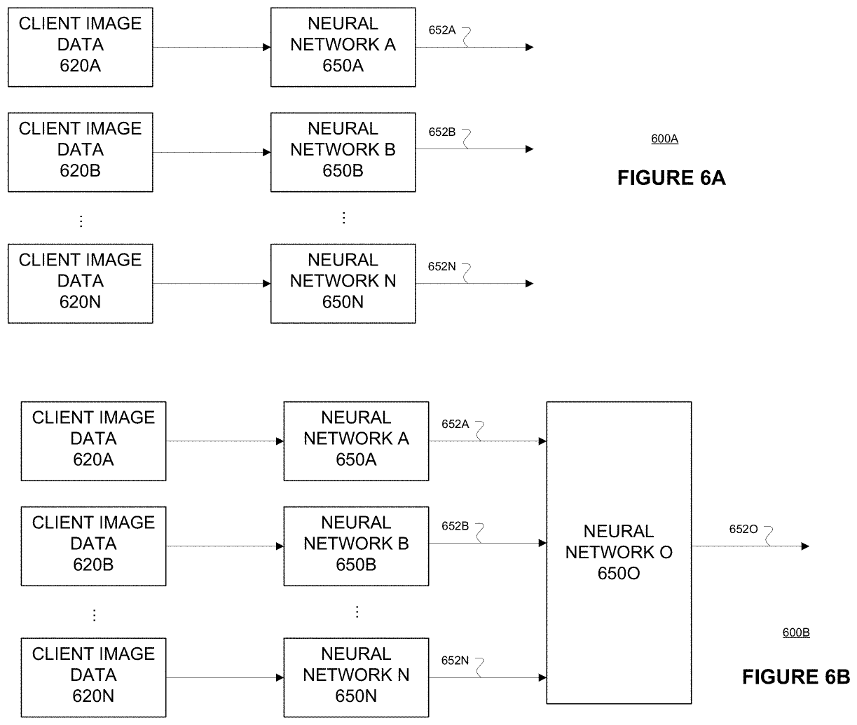

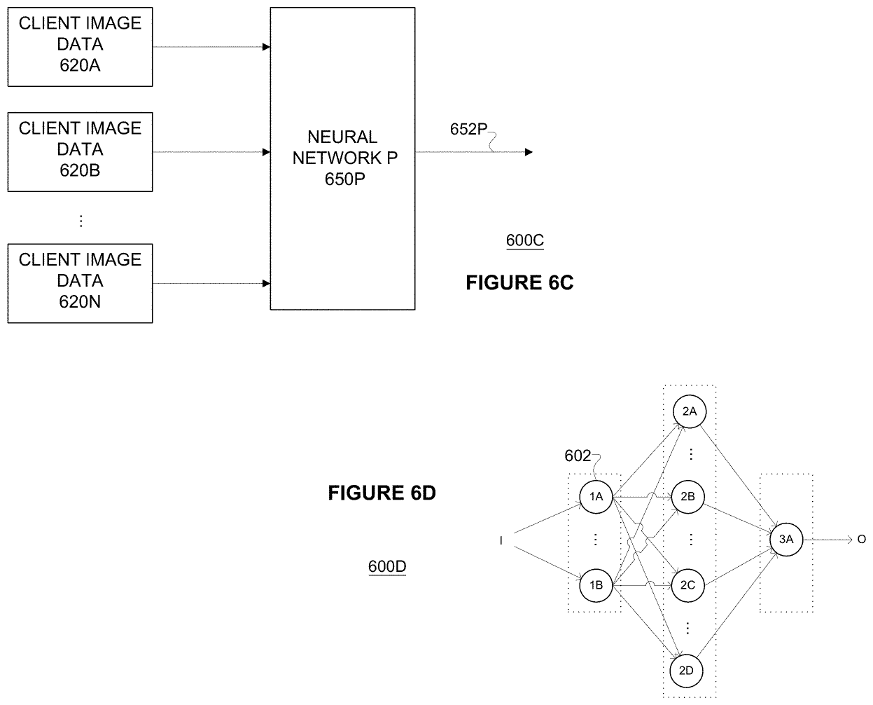

The service provider SPCI-app of a device 320A, may employ neural logic, AI, or machine learning (whether implemented locally or via call to remote computation resources) to analyze the infrared image of the client to determine their temperature (such as via FIGS. 6A-6D described below), and/or to analyze all health screening information captures by device 320A to make an assessment of whether the individual's health condition is satisfactory for an in-person service appointment. The service provider SPCI-app of a device 320A may forward the image(s) to the SPCI server 50 to employ neural logic, AI, or machine learning to analyze the infrared image of the client to determine their temperature and report same to the service provider SPCI-app of a device 320A. In an embodiment, the SPCI server 50 may forward the image(s) to an expert system 70B to analyze the infrared image of the client to determine their temperature and report same to the service provider SPCI-app of a device 320A.

Then a service provider SPCI-app of a device 320A may automatically detect the client identity based on their voice or image(s) stored in a database (activity 512) and may use neural logic, AI, or machine learning to do so, employ the SPCI server 50, or expert system 70B as described in relation to determining their temperature.

In some embodiments, the client's identity and proximity to device 320A may be determined via short range radiofrequency communication with a mobile user device 20A. For example, device 320A and user device 20A may exchange data using Bluetooth communications. Alternatively, provider device 320A may include a wireless beacon, detectable by a SPCI-app mobile application operating on user device 20A, causing device 20A to report its proximity to device 320A via, e.g., a network communication therebetween.

If the client identity is detected, a custom welcome page for the client may be provided on the device 320A (activity 516) via the SPCI-app. Otherwise, the client may be requested to register with the system 320A and download the SPCI-app on their personal device 20A-D. Their image and voice data may be stored in a database based on their registration (activity 514).

Then the client may be able to check in for a service session (activity 518, 524), and/or select an available appointment (walk-in, activity 522). Then if there is time before their schedule session (activity 526), the device 320A via SPCI-app may provide related session product sales and related session education (activity 528). Once the client has left the area of the device 320A, which its camera 322A-C may detect (activity 532), the device 320A may reset the screen to a preset display (such as shown in FIG. 2E) to protect the client's privacy (activity 534).

Another client 21B may check in via their personal device 20A. In an embodiment, an SPCI-app on their device 20A may be location aware and provide different functionality (such as check in, health screening (temperature check for example), related session product sales, and related session education) when determined to be near a service provider location 420. Further, a service provider 31A may perform similar activities for an incoming or departing client 21A-D via an SPCI-app on their device 30A.

A client 21C in a room 430B awaiting or completing a service session via their device 20B or an interactive television and keyboard 331B may similarly be able to review session options and education about the options, SPCI-history, payment options, and session related products (for sale). A service provider 31E in a room 430D via a portable computer 30E (tablet or the like), interactive television 330C, or the client's device 20D may also be able to present session options and education about the options, SPCI-history, payment options, and session related products (for sale) to the client 20D via SPCI-app running on devices 208, 20D, 3308-C. Service providers 318, 31C via an SPCI-app on their devices 308-C may be able to review session status for clients, completed consents, payment status, update other provider's 31A, D, E schedules and direct or control the SPCI-apps or information being provided or shown on various devices 320A, 330A-C, 30A-E in the location 420. Similarly, service provider 31D via their device 30D may be able to review session status for clients, completed consents, payment status, update other provider's 31A-C, E schedules and direct or control the SPCI-apps or information being provided or shown on various devices 320A, 330A-C, 30A-E in the location 420. Service provider 311) in room 430E may also be able to control the display on device 330A to show options, SPCI-history, payment options, and session related products (for sale) to a client 20A-D when in their room in an embodiment.

A variety of mechanisms may be provided for interaction between clients and service provider device 320A. In some embodiments, device 320A will include a large touch-screen and/or keyboard for touch-based interactions. In some embodiments, device 320A will include a speaker to convey audible instructions, and a microphone coupled with voice recognition and natural language processing capabilities, to enable voice interactions without requiring an individual to actually touch kiosk 320A. In some embodiments, service provider device 320A may communicate wirelessly with a user device 20 so that a client may type, tap or swipe user device 20 (e.g. via a SPCI-app implemented thereon) to transmit communications to provider device 320A, thereby minimizing the role of provider device 320A as a shared contact point and potential source for transmission of infectious agents.

Aspects of the in-person service platform are described herein with respect to a user device, client device or patient device (e.g. devices 20A, 208). In some embodiments, such personal electronic devices may be owned by the patient and brought by them into the service environment. Use of the patient's own device may be desirable for, e.g., minimizing needs for patients to touch common surfaces and thereby reduce risk of spread of infectious disease. However, in other embodiments, the devices (e.g. devices 20A, 20B) may be provided to a patient, by a service provider, for temporary use during the in-person visit. For example, an in-chair tablet may be provided, implementing the SPCI-app, in order to facilitate viewing of content such as patient education materials, treatment plans, completing check in procedures, signing consents and approvals, viewing of advertisements or promotional materials for relevant products and services, and the like.

### Exemplary In-Person Service Session Architecture

FIG. 21) is a block diagram of an architecture 1001) that may be employed for an SPCI in-person session activities in an embodiment, such as for the environment shown in FIG. 1B. Architecture 100D may also include a SPCI server 50 that provides data to SPCI-apps on devices 20A-B, 30A-E, 320A, and 330A-C, such as webpage data for web applications or data for other applications (such as SPCI specific client or provider applications) (SPCI-app). The SPCI server 50 may also communicate with other providers via their systems 40A, 70C-D, 80A-B to receive, store, and process session related data. The SPCI server 50 may include its own databases 54, 56, modules 58, 59, and interface web-server 52A to process session related data and communicate with devices 20A-B, 30A-E, 320A, and 330A-C and systems 40A, 70C-D, 80A-B in architecture 100D.

As noted above, a client device 20A, 20B SPCI-app may provide different options as function of its detected location or user-client selection. Client location within a service environment may be determined in a number of ways, such as using wireless beacon technology, location services, manual selection, QR code scanning by a client device 20A of QR codes located throughout a service provider's environment, or the like.

For example, client device 20A when located in the check-in area or room 430A SPCI-app may provide a check-in, service review, and health check options (as shown in FIG. 2D). In an embodiment, the health check option for a user-client device 20A may include asking the client a series of health status questions and using the device's camera to determine the clients body temperature via infrared imaging. The check in option may enable the client to confirm selected services, consent to the selected services, consider additional related services, and pay for selected services in an embodiment.

For a client's device 20B at a different location (such as in room 430B waiting to receive services), a SPCI-app may provide information-education about services and related services, options to purchase related products or services, in addition to the ability to authorize or consent to various services or options. In an embodiment, the SPCI-app for a client device may be programmed to provide different options-functions based on the specific location of the device 20A-B within a service provider environment 420. In an embodiment, a service provider 31A-E via an SPCI-app on their device 30A-30E may be able to control the options a client's device 20A-B presents at different locations in their environment 420.

Similarly, a service provider 31A-E via an SPCI-app on their device 30A-30E may be able to control the options presented on their service devices 320A, 330A-330C at different locations in their environment 420 as shown in FIG. 2D.

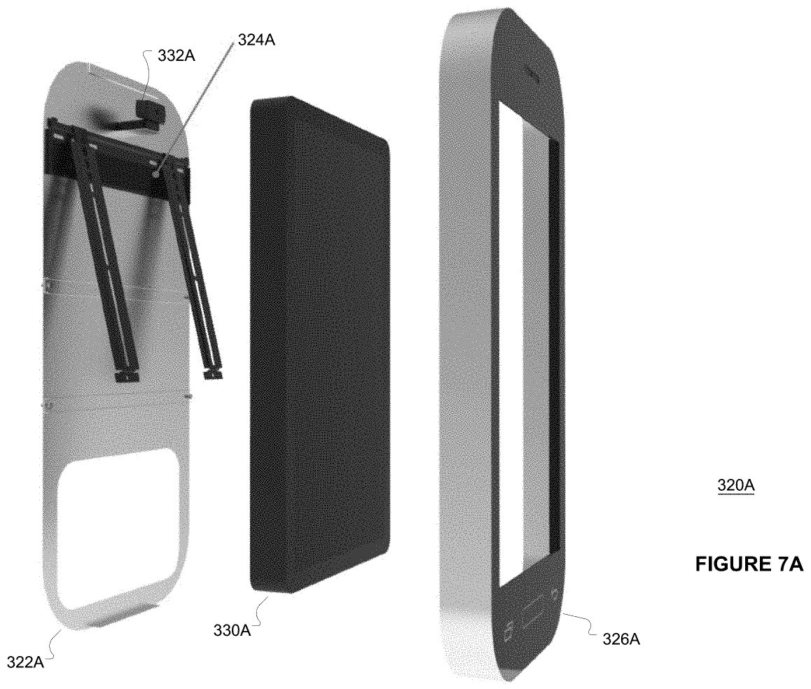

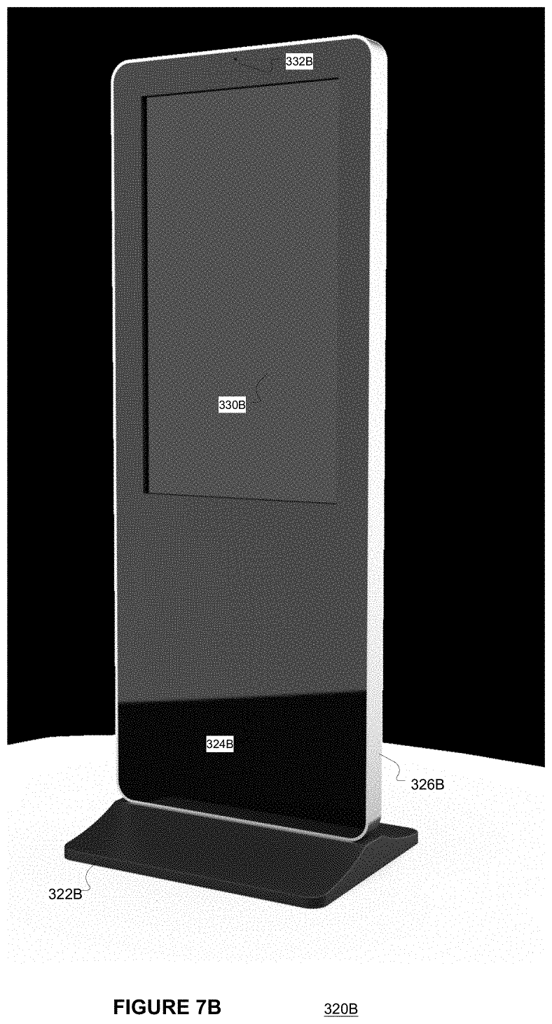

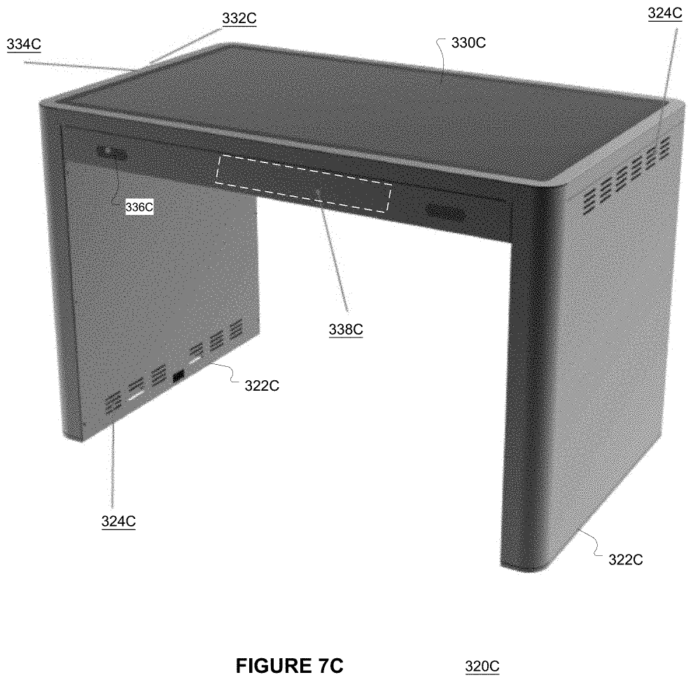

As briefly mentioned hereinabove, service provider device 320A in the waiting or check in room 430A of environment 420 may be an interactive television or custom configuration kiosk as shown in FIGS. 7A-7C. As shown in FIG. 7A device 320A may be a wall mounted system including a separate camera 332A, wall mount 322A, coupling arms 324A for holding an interactive device 330A and a shroud or cover 326A. As shown in FIG. 7B, the system 320B may be a free-standing unit including a pedestal 322B, storage area 324B, separate camera 332B, interactive device 330B, and case 326B. As shown in FIG. 7C, the system 320C may have a table configuration with an interactive device 330C coupled to legs 322C with venting 324C, a case 334C including a separate camera 332C, speakers 336C and computing unit 338C. The computing unit 338C: may run the service provider SPCI-app in an embodiment and communicate images and receive inputs from the interactive device 330C. In an embodiment, the interactive devices 330A-C may run the service provider SPCI-app.

FIG. 2E is an exemplary interface-display 210A that a SPCI-app may provide on a service provider device 320A-C. As shown in FIG. 2E, the display 210A may include a banner 212A showing the service provider's name, company, or service mark(s). The display 210A may also include an educational section 214A showing multimedia about various services that the service providers 30A-E may provide. The display 210A may also include a section 215A that provides information about the service providers 30A-30E such as experience and credentials. The display 210A section 216A may enable a client 21A to select one of several options. The options may include educational information about services, tips for the client, additional services available, and payment options. The display 210A may also include more service provider banners 218A and advertising content section 222B in an embodiment.

As shown in FIG. 2D, a service provider device 320A may enable a client 21A to access several functions or options including check-in, health check, information about services and related services, and third-party related products and services as described above. The separate camera 332A-C may include infrared capabilities and be able to detect the body temperature of a client 21A-B adjacent to it. As shown in FIG. 2D, the other service provider devices 30A-30E and 330A-330C may provide various other options or functions for both the clients 21A-D and service providers 30A-E to select. For example, a service provider 31E may employ a handheld portable computer (device) such as a tablet 30E (i.e. an in-chair tablet) that it may use to discuss and provide services and options for the client 21D. As shown in FIG. 2D, a service provider device 30D may enable the provider 30A to view their current schedule, remote service requests (view critical service queue). Their service schedule may also indicate the service(s) that the client has requested or authorized so they may prepare accordingly.

As shown in FIG. 1A and discussed with reference to FIGS. 1B-1C and 2C-2D, the SPCI-app for a client device 20A may be employed by a client 21A for all services needs including remote and in-person. In an embodiment, a SPCI-app and SPCI server 50 may employ the algorithm 540 shown in FIG. 5E. As shown in FIG. 5E and discussed above, in an embodiment, the SPCI-app of a client device 20A-B may use its location information to determine when the device 20A-B is near a service provider environment 420 (activity 542) and to receive in person services. When near a service provider environment 420, the SPCI-app of a client device 20A-B may enable a client to view in-person options (activity 544) and provide various options for in-person service as discussed above when elected (activity 546 and 548) in an embodiment. As noted, the in-person options may vary based on the location of the client 21A within a service provider environment 420.

In an embodiment, when a client's device 20A-D is not near a service provider 31A-E location, the SPCI-app may provide remote service options including the ability to view their SPCI-history (activity 552 and 554), selecting a remote service session (activity 556), request an in-person service session (activity 558), and view their current service schedule (for already request in-person or remote services) (activities 562, 564). When a client 21A via the SPCI-app requests a remote service session, they may be able to request a non-critical service session (activities 566, 568), a critical service session (activities 572, 574), and other service activities (activities 576, 578).

As described above, a SPCI-app on a client device 20A-D, service provider device 30A-E, 320A, 330A-C, SPCI server 50, or an external expert system 70B may use neural logic, artificial intelligence, or machine learning to determine the identity or temperature of a client 21A-D. The same neural logic, artificial intelligence, or machine learning may be used for automated identification of other states or conditions of a client 21A-D or an item they want serviced (for example view images of clients' teeth and compare to stored x-rays to detect an anatomical change requiring service, view images of tires of a client's vehicle and determine the type, size, and condition (under-over inflated, tread depth too low, damaged side wall, un-natural shape) of tires that may need replacement or service). For example, such analysis may be performed prior to contacting a service provider 31A-E via their SPCI-app on their device 30A-E to conduct a remote interactive session between a service provider and a client so their interaction may be more effective and efficient for both parties.

Accordingly, an embodiment may use neural logic, artificial intelligence, or machine learning to provide triage or concierge services or analysis before, during, after remote or in-person services. For example, an embodiment ofa SPCI-app on a client device 20A-D, service provider device 30A-E, 320A, 330A-C, or SPCI server 50 may employ a neural network architecture 600A-D as shown in FIG. 6A-D to process client images according to various embodiments.

As shown in FIG. 6A, each client image represented by data 620A-N may be coupled to a separate neural network system 650A-N. In such an embodiment, a neural network system A-N 650A-N may be trained to respond to particular image data (generated, received, and position) based on one or more developed logic/model/procedure (L/M/P). The neural network system A-N 650A-N outputs 652A-N may be used individually to analyze the image data. In another embodiment, the neural network systems A-N 650A-650N may be coupled to another neural network system O 6500 as shown in FIG. 6B. The neural network architecture 600B may enable neural network systems A-N 650A-N to process image data and neural network system O 6500 to process the neural network systems A-N 650A-N outputs 652A-652N. The neural network system O 6500 may then determine attributes about the provided image(s) based on neural processing of combined neural processed image data. The neural network system O 6500 may be able to make decisions based on a combination of different image data from different image data A-N 620A-N (where the images could be prior client image(s) or other reference image(s)) and based on one or more developed L/M/P, making the neural network system O 6500 more closely model a service provider professional 31A-E, which may consider many different images in addition to reference image(s) when formulating an action or decision.

In a further embodiment, a neural network architecture 600C shown in FIG. 6C may employ a single neural network system P 650P receiving and processing client image data, which could also be sensor data from a plurality of sensors such as cameras 332A-C. Similar to the neural network system O 6500, the single neural network system P 650P may be able to make decisions based on a combination of different sensor-image data, making the single neural network system P 650P also more closely model a service provider professional 31A-E, which may consider many different image(s) in addition to the client image(s) when formulating an action or decision. In an embodiment any of the neural architectures 600A-C may employ millions of nodes configured in various configurations including a feed forward network as shown in FIG. 6D where each colunm ofnodes 602 1A-IB, 2A-D, 3A, feeds the next right column of nodes. The input vector I and output vector O may include many entries and each node may include a weighted matrix that is applied to the upstream vector where the weight matrix is developed by the training database 56 and training systems 50.

Different sets of neural networks 600A-600D may be trained/formed and updated (evolve) for a particular activity ofa service analysis-procedure. One or more L/M/P may be developed based on availability of image data to perform a particular activity of a session procedure. The different sets of neural networks 600A-600D may be trained/formed and updated (evolve) for a particular activity of a session analysis-procedure based on the developed one or more LIM/P.

In an embodiment, all client data including images and any analysis, past service interactions, tests, communications, and service(s) provided (SPCI-history) may be stored by the SPCI server 50 or off-site system 40A. Such storage may enable a SPCI-app to retrieve past service interactions, tests, analysis, communications, and completed service(s) to enable a service provider 31A-E to be able to provide enhanced or more beneficial future service(s) for a client 21A-D. In addition, a client 21A-D may have access to their entire SPCI-history via an SPCI-app on their device 20A-D.

As noted, a service provider 31A-E may be a medical provider and the client 21A-D may be a patient in SPCI architecture 10, 400, 420, 100C, 100D. FIG. 1C is a block diagram of another service provider-client interactive (SPCI) architecture 10 according to various embodiments. As shown in FIG. 1C, SPCI architecture 10 may include a plurality of service provider-client interactive (SPCI) client devices 20A-20D, one or more service provider-client interactive (SPCI) provider devices 30A-30E, one or more service provider-client interactive (SPCI) servers 50A-50B, one or more cloud-blockchain systems (CBCS) 40A-40B, one or more remote interactive SPCI admin systems 60A-60B, one or more other providers systems 70A-70D, and one or more payment systems 80A-8. As noted in FIG. 2C, a payment system 80A may be an electronic payment processing system and payment system 80B may be a third-party payor system such as an insurance company system. The other providers systems 70A-70D could be a voice analysis system 70A, an expert system 70B, a service information-education system 70C, and a third-party sales system 70D in an embodiment.

In an embodiment, the CBCS 40A-40B may include certificate authority entities that create or issue digital certificates. In an embodiment, the CBCS 40A-40B may employ cryptographically linked blocks in an open, distributed ledger termed blockchains and hereinafter systems 40A-40B are referred to as CBCS although they could be any cryptographic provider, system, software, or entity and provide cloud services.

In an embodiment, a client device 20A-D, a provider device 30A-30E, a CBCS 40A-40B, an admin system 60A-60B, other provider system 70A-70D, and payment systems 80A-80B may communicate with a SPCI server 50A-50B via one or more networks 16 where the networks may be local wired or wireless networks or a network of networks such as the Internet.

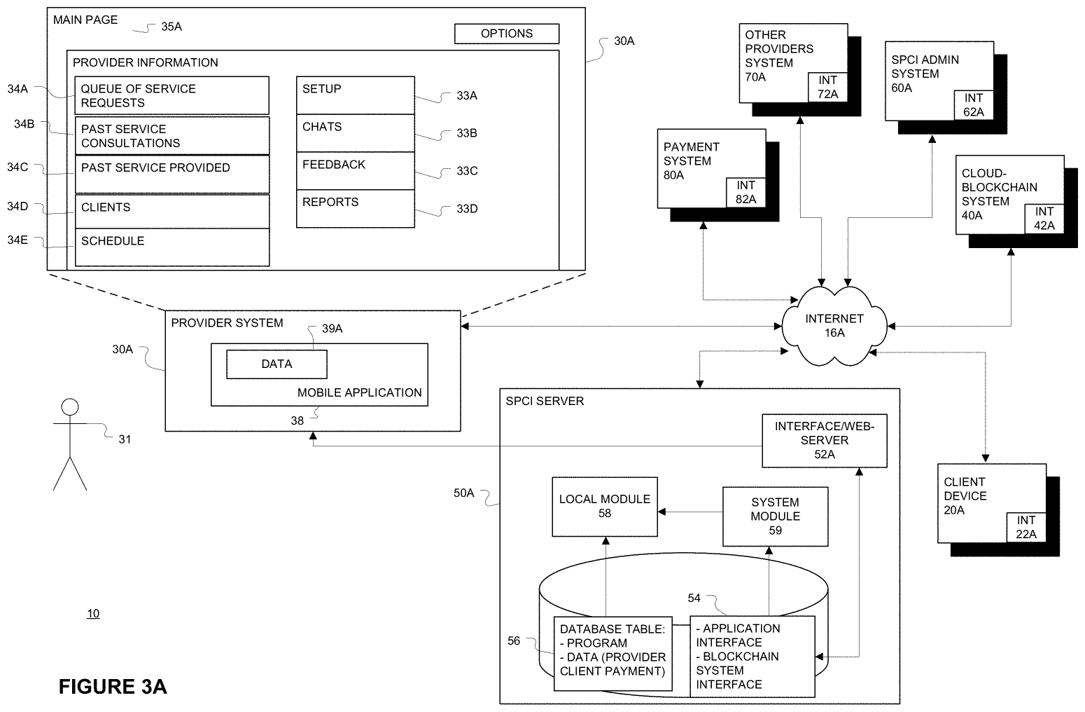

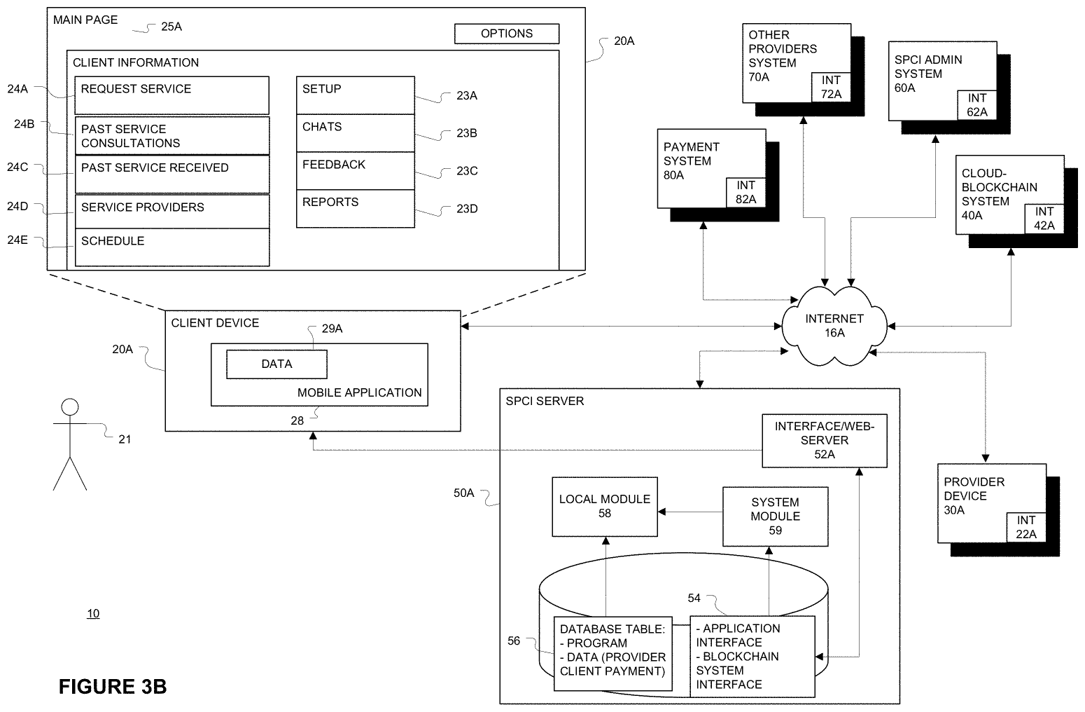

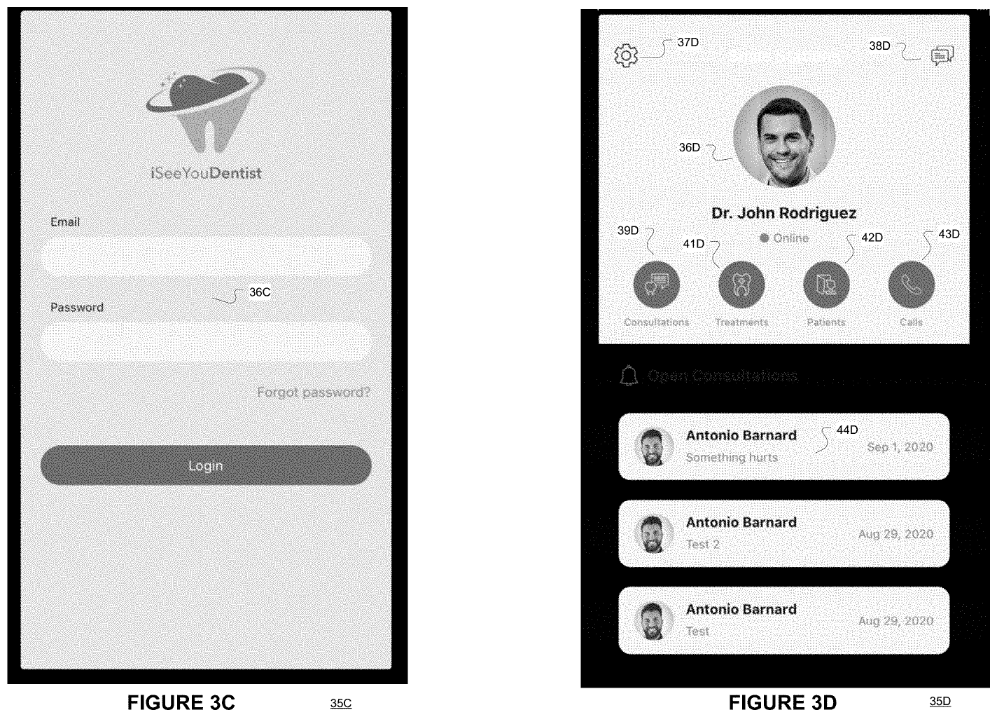

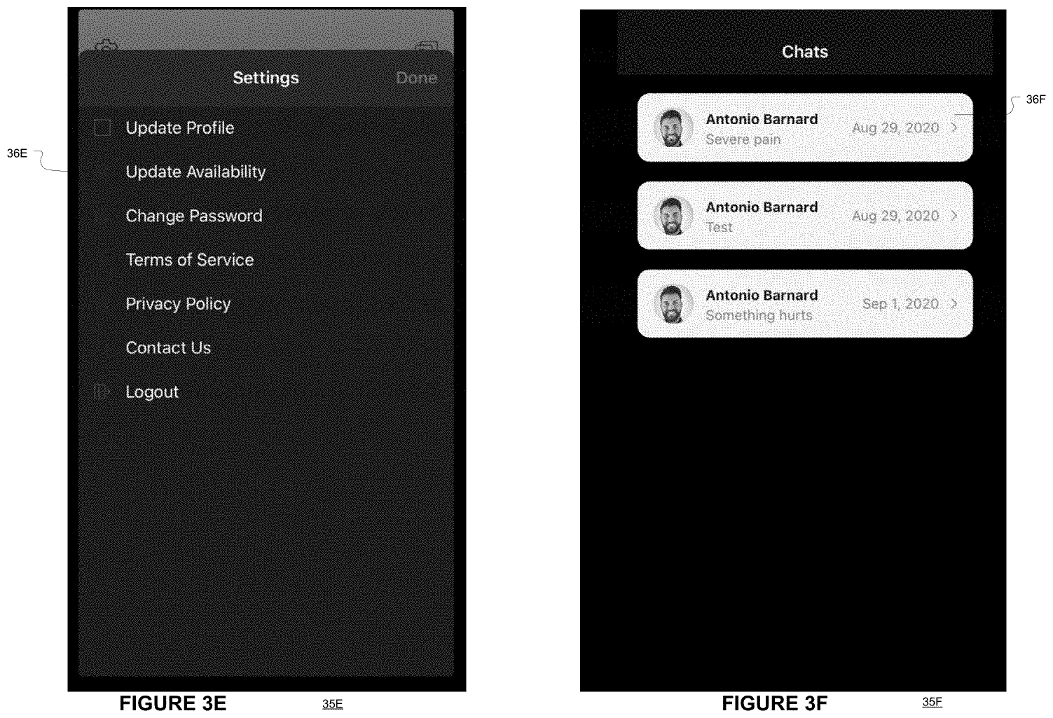

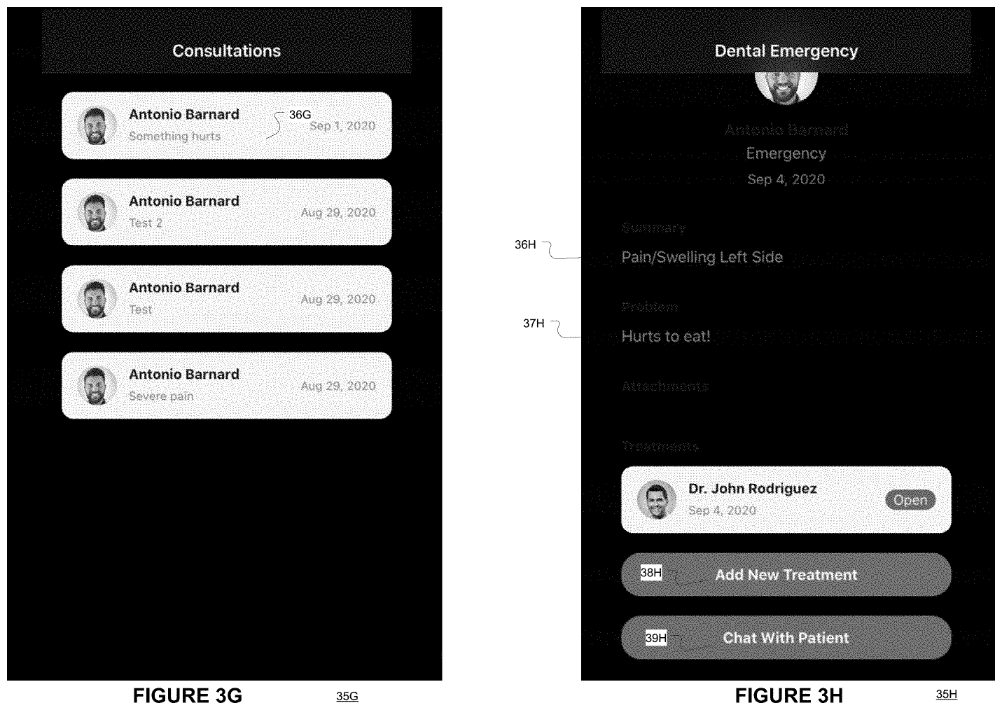

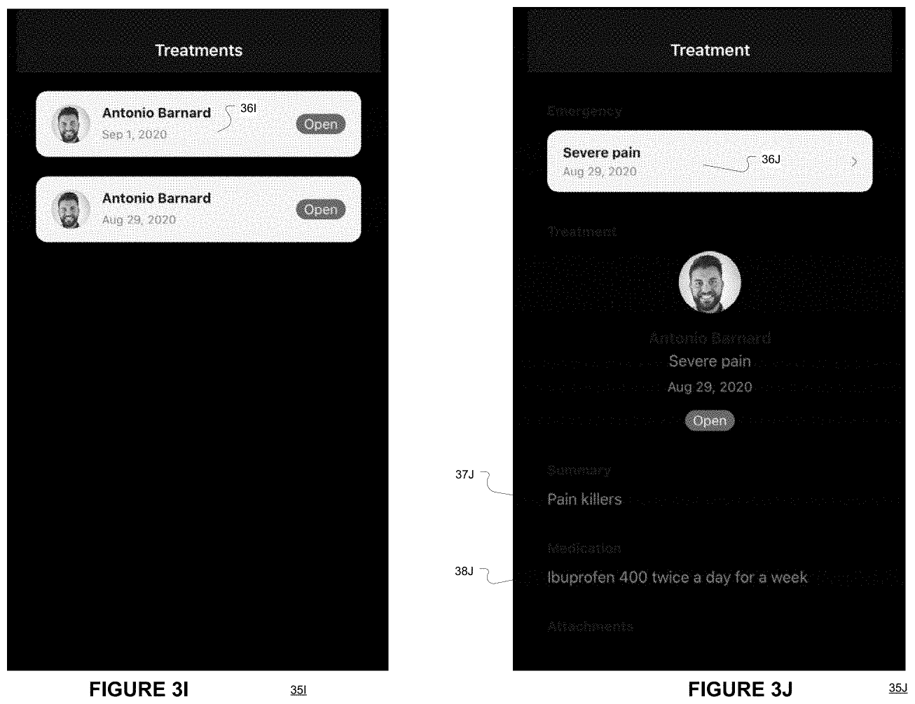

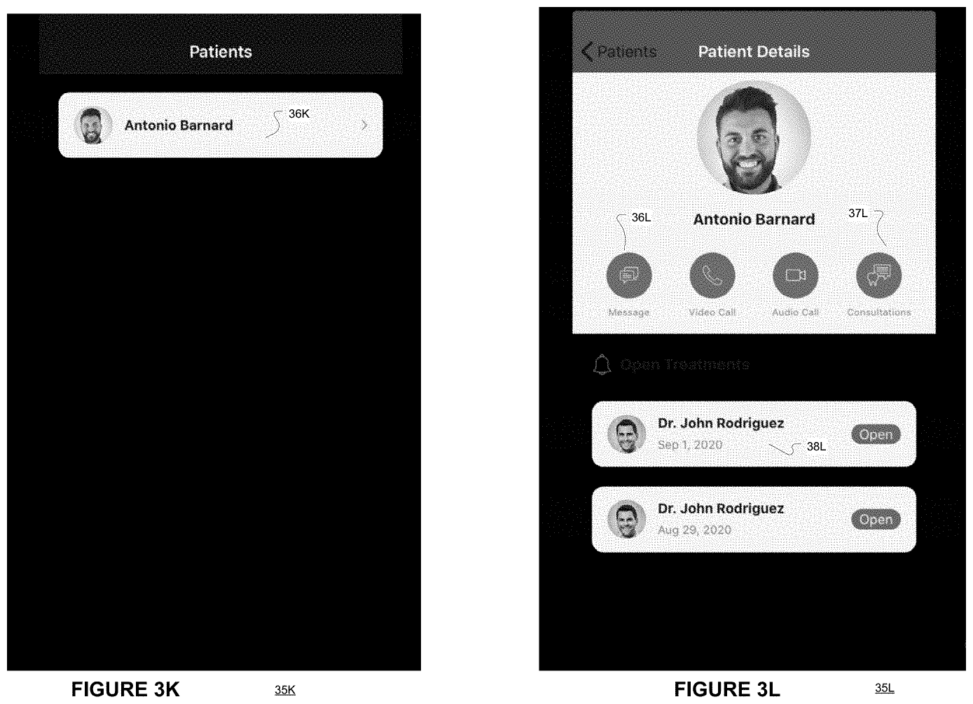

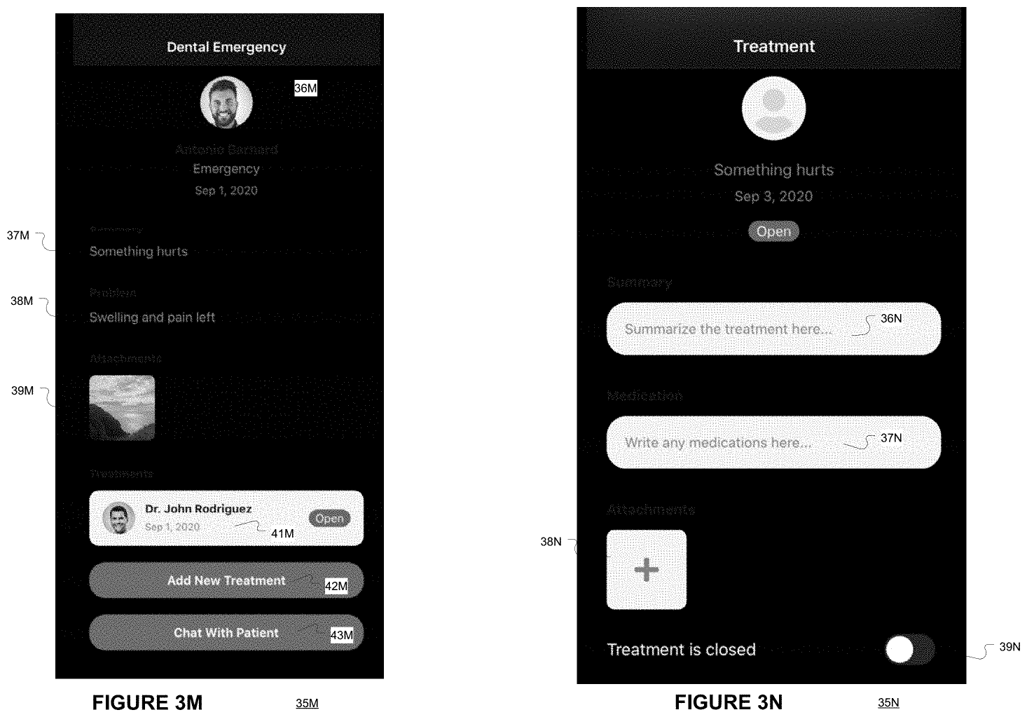

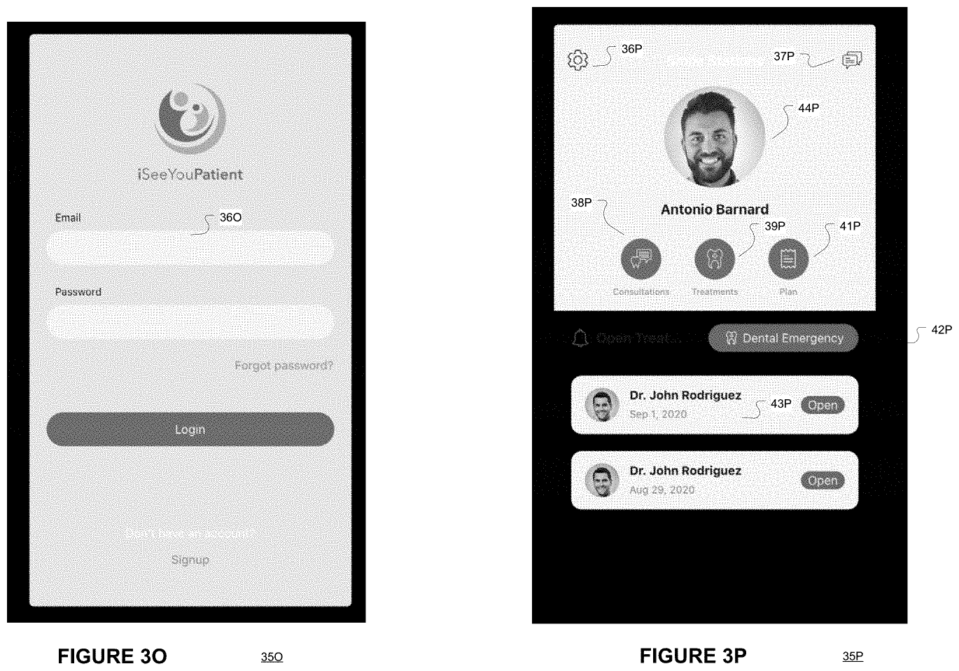

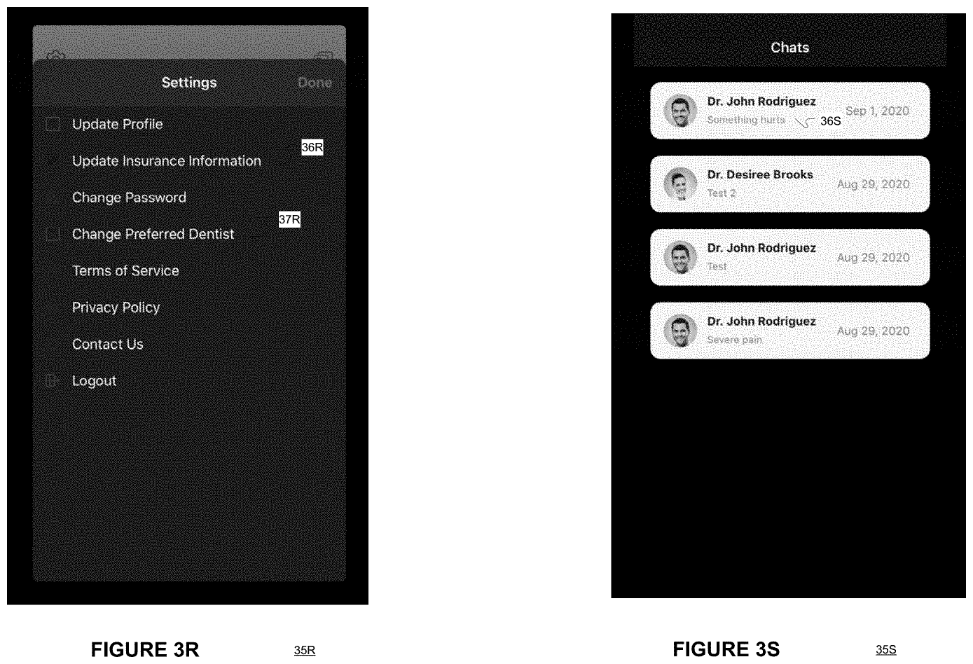

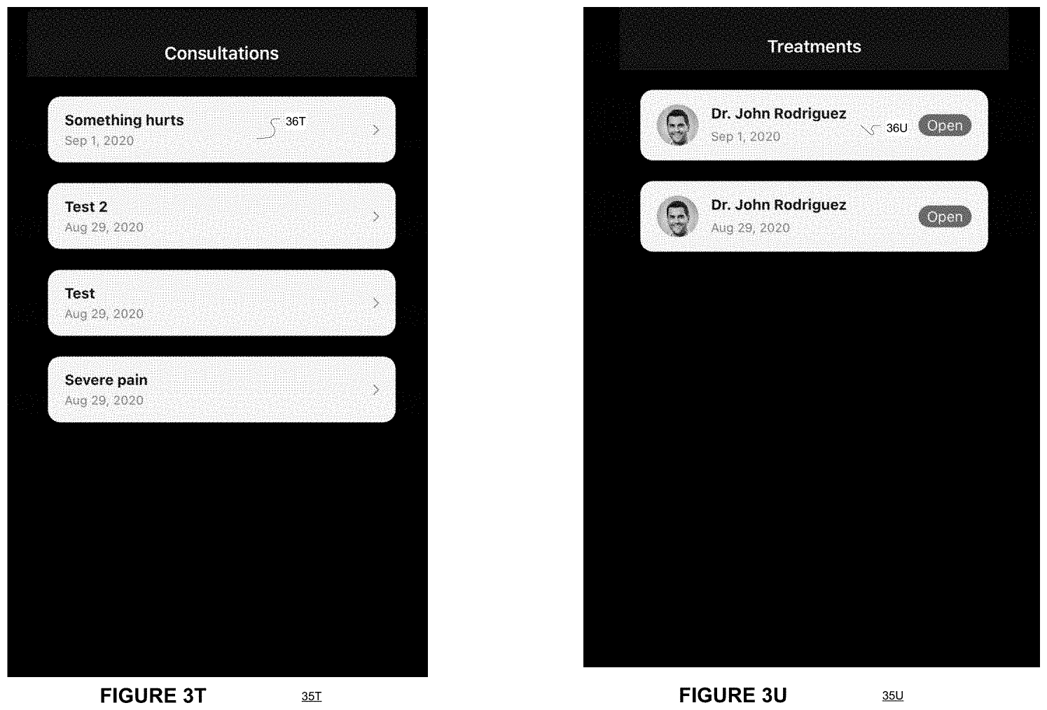

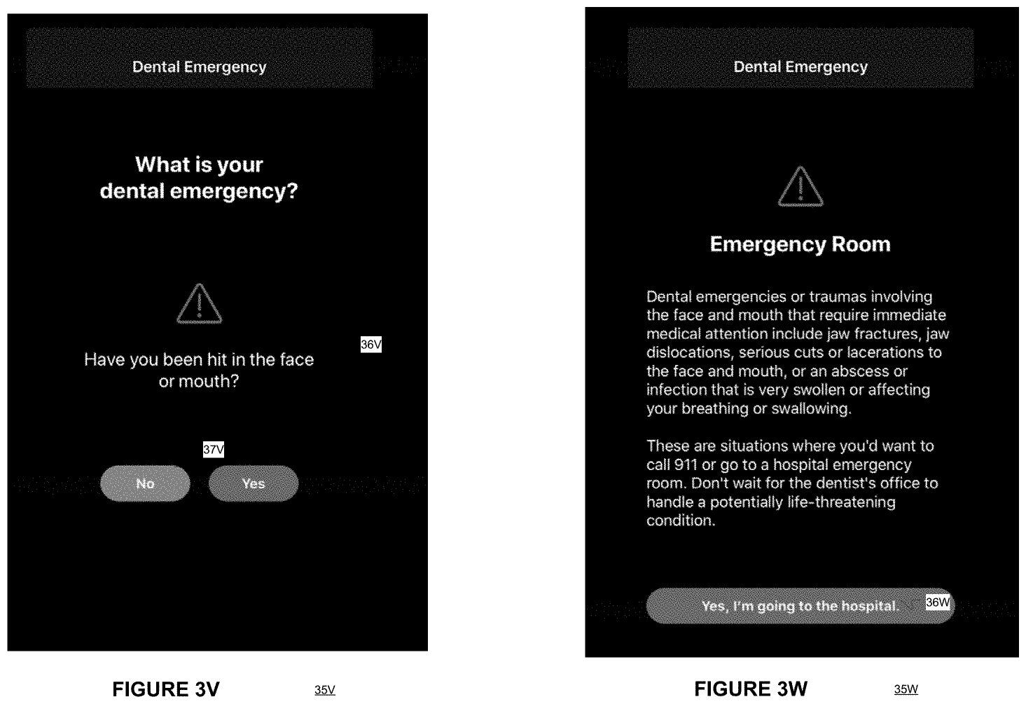

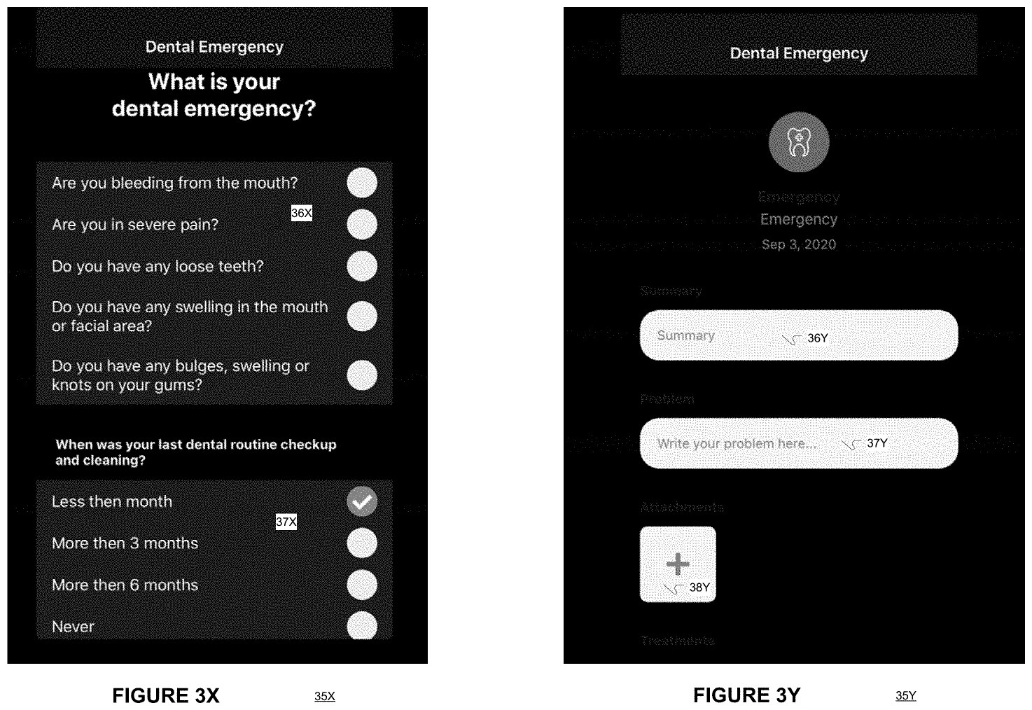

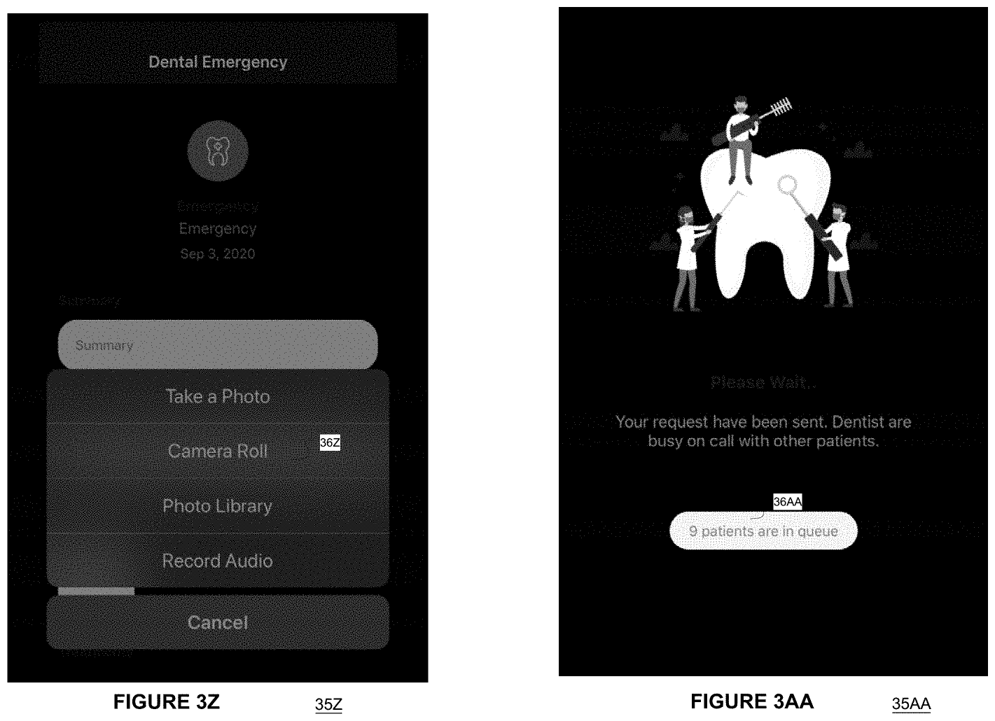

In an embodiment, a client via a client device 20A-D (hereinafter 20A for simplicity) via a local SPCI application, real-time SPCI application, or web-based SPCI interface (such as browser) (herein after client SPCI-app) may interact with a SPCI server 50A (such as via main user interface 25C in FIG. 3P and 25A in FIG. 3B of a client SPCI-app). Similarly, a provider via a provider device 30A-30E (hereinafter 30A for simplicity) via a a local SPCI application, real-time SPCI application, or web-based SPCI interface (such as browser) (herein after provider SPCI-app) may interact with a SPCI server 50A (such as via main user interface 35D in FIG. 3D and 35A in FIG. 3A of a provider SPCI-app). A SPCI administrator via an admin system 60A-60B (hereinafter 60A for simplicity) via a local SPCI application, real-time SPCI application, or web-based SPCI interface (such as browser) (herein after admin SPCI-app) may interact with a SPCI server 50A.

As noted above, a SPCI server 50A may communicate with a payment system 80A (such as Stripe®, Venmo®, credit card processor, bitcoin, Paypal®, EFT processor, or others) to process payment to a provider device 30A for services provided (such as provided services show on pages 35M-N in FIGS. 3M-N). In an embodiment, SPCI server 50A may communicate with a 3^{rd} party payor system 80B (such as an insurance provider system) to request payment to a provider device 30A for services provided that may be covered by the 3^{rd} party payor. The CBCS 40A may be used to create tokens related to payment and store all related activity (such as services requested and performed and related communications) using open, distributed ledgers, i.e., blockchains including other provider systems 70A.

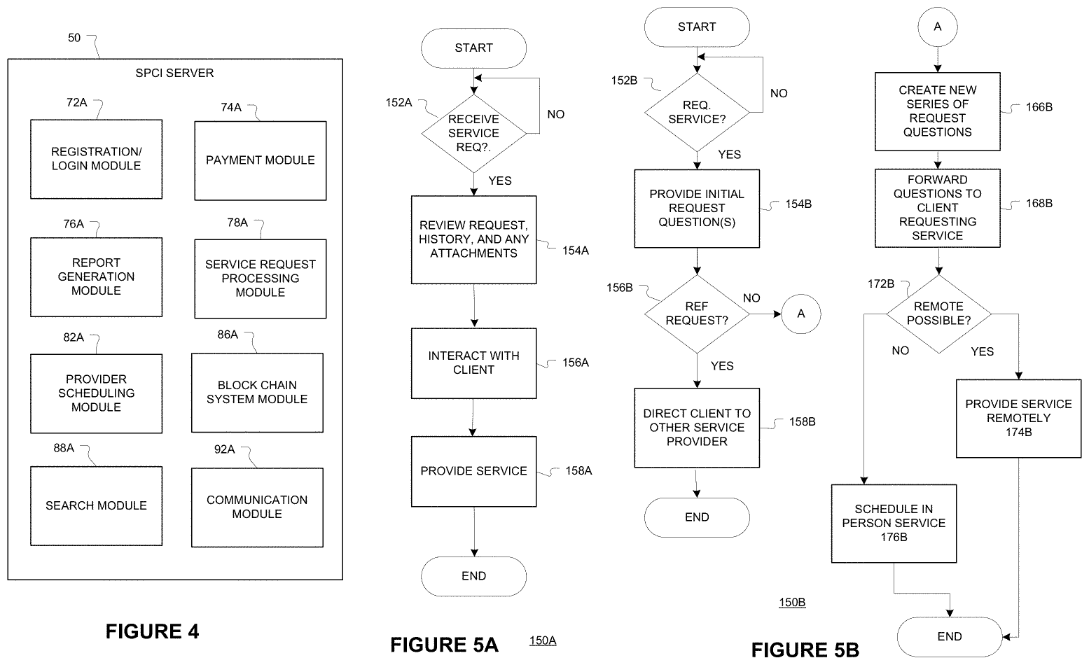

In an embodiment, a SPCI server 50A may communicate with a CBCS 40A to store client and provider information and activity including payment, login, security, requested services, provided services, communications, and other information in a blockchain via a block chain system module 86A (shown in FIG. 4). In an embodiment, another provider system 70A may perform service-related activities as directed by a service provider for a client. For example, where a provider is a medical services provider, another provider system 70A may be a prescription fulfillment system coupled to a pharmacy, testing system coupled to a medical laboratory, expert system, voice analysis system, 3^{rd} party sales system, and service information-education system. In an embodiment, a SPCI server 50A may also communicate with another provider system 70A to forward services requests from a provider device 30A.

In an embodiment, the CBCS 40A-40B (hereinafter 40A for simplicity) may employ cryptographically linked blocks in an open, distributed ledger termed blockchains in addition to digital certificates from cryptography certificate authority entities. A CBCS system 40A employing blockchains (hereinafter CBCS 40A for simplicity) may generate digital certificates, identifiers (IDs), or tokens than are unique and tied and copied/distributed to a plurality of systems 40A to secure the digital certificates, identifiers (IDs), tokens, client information, provider information, services requested and performed and related communications, and payments. Similarly, in an embodiment any updates associated with SPCI architecture 10, 100C, 100D, 420, 400 such as the client information, provider information, services requested and performed and related communications, and payments, may be distributed across many blockchain systems 40A to prevent corruption of SPCI architecture 10, 100C, 100D, 420, 400 data and provide a secure and reproducible record or ledger of all activity along with the source of such changes. In an embodiment, all client information, provider information, services requested and performed and related communications, and payments may be assigned a unique token that is created and stored by a CBCS 40A

In an embodiment as shown in FIGS. 3A and 3B, a SPCI server 50A may include an network interface/web-server/software server 52A where the server 52A may be configured to communicate messages, graphical user interfaces, virtual reality (VR), augmented reality (AR), software, data for applications on a provider device 30A, a client device 20A, admin system 60A, CBCS 40A, and other providers systems 70A via their interfaces 32A, 22A, 62A, 42A, and 72A.

In an embodiment, a provider device 30A, client device 20A, and admin system 60A may host a local SPCI application, real-time SPCI application, or web-based SPCI interface (such as browser) (herein after admin SPCI-app) 28 and 38, each of which may maintain a local store of data 29A and 39A, respectively. The web-based SPCI interface may include a web browser such as Internet Explorer, Edge, Safari, Netscape, Chrome, Firefox, or Opera, VR system, or AR system where the SPCI-app may communicate with the SPCI server 50.

In an embodiment, a provider device 30A, client device 20A, admin system 60A, CBCS 40A, and other providers system 70A via their interfaces 32A, 22A, 62A, 42A, and 72A may be any computer device capable of hosting an interface that can communicate with a SPCI server 50A including via web browser application 28, 38 runtime application, VR system, AR system, application programming interface (API), or other application as noted. A provider device 30A, client device 20A, admin system 60A, CBCS 40A, and other providers system 70A may include a processor with a network interface 32A, 22A, 62A, 42A, and 72A including a server, virtual server or system, personal computer, personal data assistant, interactive television, cellular phone, video game console, or tablet computer.

In an embodiment, a SPCI server 50A may employ a web framework (WF) or web application framework (WAF) including Ruby on Rails, Java, Python, Apache, Django, or other software or architecture to provide web pages, framework, or wire frames to a provider device 30A, a client device 20A, and an admin system 60A. A SPCI server 50A may also employ Software as a Service (SaaS) to provide data and executable instructions (an SPCI-app) to a provider device 30A, a client device 20A, and admin system 60A. In an embodiment, webpages may be built using on a Rudy on Rails framework or other web frameworks. In an embodiment, a provider device 30A, a client device 20A, and an admin system 60A may host an SPCI-app operating natively where the application communicates data with the SPCI server 50A (such as a SPCI application downloaded from an application provider or provided by the SPCI server 50A).

A provider device 30A, a client device 20A, and an admin system 60A may use various operating systems including Microsoft operating systems (Windows), Linux, Mac OS X, Android, WEB OS, and others to run a SPCI-app. In an embodiment, a SPCI server 50A may provide SPCI-app to run natively on a client device 20A. Such an interface may enable VR system, or AR systems to operate on a client device 20A.

FIG. 2A is a diagram of communications 100A between a client device 20A, a provider device 30A, a 3^{RD} party payer system 80B, a SPCI server 50A, and a cloud-blockchain system (CBCS) 40A according to various embodiments. FIG. 2B is a diagram of communications 100B between a client device 20A, a provider device 30A, a payment system 80A, a SPCI server 50A, and a cloud-blockchain system (CBCS) 40A according to various embodiments.

In architecture 10, a client 21A or a provider 31A may be required to register with/login to a SPCI server 50A via a SPCI-app on their device 20A, 30B—communications 102A-B, 112A-B of FIGS. 2A and 2B via registration/login module 72A (FIG. 4) according to various embodiments. As shown in FIGS. 2A and 2B, a SPCI server 50A may receive a registration/login request (communication 102A-D, 112A-D), such as via interfaces 35C and 350 selections 36C and 360 (from SPCI-apps) shown in FIGS. 3C and 30, forward initial client 21A and provider 31A information to a CBCS 40A for encrypted and distributed storage (communications 104A-B, 114A-B) and request a unique ID for the client 21A and the provider 31A in an embodiment. In an embodiment, a client 21A may have a login created by another provider such an insurance provider or via a portal from the 3^{RD} party payor system 80B for use in SPCI architecture 10, 100C, 100D, 420, 400.

This unique ID for a client 21A or a provider 31A may allow them to create a secure history (SPCI-history) in the architecture 10, 100C, 100D, 420, 400 so another client 21B-D or another provider 31B-E may view their SPCI-history (as permitted) as they use architecture 10, 100C, 100D, 420, 400 over time. In an embodiment, the unique ID or IDs for a client 21A or a provider 31A may include one or more blockchain tokens that are uniquely and securely associated only with the client 21A and the provider 31A. The SPCI server 50A may receive and store the generated unique ID(s) or token(s) for the client 21A and the provider 31A.

Once registered, a client 21A or a provider 31A may login into a SPCI server 50A via a client SPCI-app or provider SPCI-app on their devices 20A, 30A, thereafter using secured protocols. In an embodiment, a client 21A or a provider 31A may be able to create a profile and add images (36D in FIG. 3D and 44P in FIG. 3P) to their profile via a SPCI-app on a client device 20A, a SPCI-app on a provider device 30A.

Once registered or logged into a SPCI server 50A, a SPCI server 50A may generate and provide/forward a main page 25A, 35A (communications 106A-B, 115A-B) to a SPCI-app on a client device 20A, a SPCI-app on a provider device 30A via a network 16, such as the graphical user interface screens 25A, 35A shown in architecture 10 in FIG. 3B and FIG. 3A and interface screens 35P, 35D shown in FIGS. 3P and 3D.

As shown in FIGS. 3A and 3B, a SPCI server 50A may include a network interface/web-server 52A, application interfaces and blockchain system interfaces/protocols 54, provider and client tables/databases 56, a local module 58, and a system module 59. The system module 59 may develop requests and processes responses from CBCS 40A, payment systems 80A-B, and other provider systems 70A-D. The local module 58 may interface with the provider and client table/database 56 and communicate data between the interface/webserver 52A and the database 56. The provider and client database 56 may include program data for the local module 58, system module 59, and interface/web-server 52A and devices 20A-D, 30A-E, 40A, 60A, 70A-D, 80A-B, 320A, and 330A-C and service-related data.

In an embodiment, the service-related data may include unique ID(s) created for the provider completed service requests and related information and client posted service requests and related information. The application interfaces and blockchain system interfaces/protocols 54 may include data associated with interfacing with the CBCS 40A and devices 20A-D, 30A-E, 40A, 60A, 70A-D, 80A-B, 320A, and 330A-C in an embodiment. As shown in FIG. 3A, a main page 35A (and FIG. 3D main page 35D) for a provider device 30A as generated by a SPCI server 50A may include the ability to view a queue of pending service requests from clients (34A and 44D) generated by SPCI server 50, view and change setup or settings (33A and 37D) via interface 35E of FIG. 3E showing settings 36E), past service consultations (34A and 39D) via interface 35G of FIG. 3G showing past consultations 36G with clients and FIG. 3H showing details of a past consultation 36H-39H), chats (33B and 38D) via interface 35F of FIG. 3F showing past chats or communications 36F with clients), past services provided (34C and 41D) via interface 35I of FIG. 3I showing past services (treatments in an embodiment) provided 36G to clients (patients in an embodiment)—FIG. 3J showing details of service provided 36J-38J (treatments to a patient in an embodiment), past/current clients (34D and 42D) via interface 35K of FIG. 3K showing clients (patients in an embodiment) 36K—FIG. 3L showing details of a client 36L-38L (patient in an embodiment), feedback from clients 33C, report generation 33D, and schedule for upcoming services to be performed for clients 34E. It is noted that chats 33B, 23B (FIG. 3B) may be any type of electronic communication between a service provider 31A and client 21A via SPCI-apps on their devices 30A, 20A including text, voice, video, virtual reality, or other. Further such electronic communications may be stored by the SPCI server 50 directly or via the CBCS 40A or other storage system. SPCI architecture 10, 100C, 100D, 420, 400 may employ various known communication platforms in the SPCI-apps such as short messaging service and 3^{rd} party applications (such as Apple® FaceTime®, Zoom® meetings, Google® meeting and others to enable “chats” between a service provider 31A and client 21A via SPCI-apps on their devices 30A, 20A.

As shown in FIG. 3B, a main page 25A (and FIG. 3P main page 35P) for a client 21A as generated by a SPCI server 50A (or SPCI-app) may include the ability to generate a service request (24A and 42P) to be processed by SPCI server 50 via a service request processing module 78A of FIG. 4. Via their SPCI-app a client 21A may also view and change setup or settings (23A and 36P) via interface 35R of FIG. 3R showing settings 36R and 37R), view past service consultations (24A and 38P) via interface 35S of FIG. 3S showing past consultations 36T with providers. Via their SPCI-app a client 21A may also view chats (23B and 37P) via interface 35S of FIG. 3S showing past chats or communications 36S with providers). Via their SPCI-app a client 21A may also view past services provided (24C and 39P) via interface 35U of FIG. 3U showing past services (treatments in an embodiment) provided 36U by service provider (dentist in an embodiment). Via their SPCI-app a client 21A may also view past/current service requests/providers (24D and 43P), feedback from service providers 23C, generate reports 23D, and their schedule for upcoming services 24E.

In an embodiment, a service provider 31A may be any individual or group that may provide services for a client 21A including a dentist or any staff member providing services to a patient in an embodiment. As noted, via page 35A and 35D, a provider 31A via SPCI-app on their device 30A may select a pending service request from a queue of service requests (from clients) (34A, 44D) (activity 152A of FIG. 5A of algorithm 150A). In an embodiment when a service provider 31A selects a service request (via SPCI-app on their device 30A), SPCI server 50 may provide interface 35M of FIG. 3M on their device 30A via an SPCI-app.

As shown in FIG. 3M, a service provider 31A via interface 35M shown on their device 30A via an SPCA-app may see an image of a client 36M, summary of request (or problem-concern) 37M, description of request (or problem in an embodiment) 38M, multimedia attachments 39M, and listing of previous treatments 41M. Via the interface 35M shown on their device 30A via an SPCI-app, the service provider may be able to review the request, history and attachments (activity 154A), interact with the client (43M—activity 156A), and select to provide new service (treatment in an embodiment—42M—activity 158A).

In an embodiment, a service provider 31A via SPCI-app on their device 30A may be able to forward a received service request 44D to another provider 30B_E (via SPCI-app on another device 30B-E or other system) for a second opinion on the processing of the service request. In addition, a service provider 31A via SPCI-app on their device 30A may be able to forward a received service request 44D to their staff or related service provider (such as another professional, assistant, manager) (via SPCI-app on another device 30B-E or other system) for at least partial processing of the service request.

When a service provider 31A selects to provide new service via SPCI-app on their device 30A, interface 35N shown in FIG. 3N may be provided. As shown in FIG. 3N, a service provider 31A via SPCI-app on their device 30A may be able to provide a summary of the service to be provided (summary of treatment in an embodiment) 36N, assign services to be conducted or provided (treatments to be prescribed or laboratory work to be done in an embodiment) 37N, include multimedia attachments 38N (such as voice, text, or other media instructions or information). The service provider 31A via SPCI-app on their device 30A may also be able to note when the requested service is completed 39N. A service provider 31A via SPCI-app on their device 30A may also be able to schedule an in-person meeting with the client. When the service provider 31A via SPCI-app on their device 30A provides treatments to be prescribed or laboratory work to be done in an embodiment, the SPCI server 50 may forward the treatments to be prescribed or laboratory work to be done to another provider system 70A for processing.

In an embodiment, a service provider 31A via SPCI-app on their device 30A may be able to provide initial steps required to complete a service request. The service provider 31A via SPCI-app on their device 30A or SPCI server 50 may refer a client 21A via SPCI-app on their client device 20A to an in-person meeting with the service provider 31A or another service provider 31A to conduct additional steps of the service request. The service provider 31A due to regulations or other factors may not be able to request or order procedures required to complete a service request for a client 21A, e.g., a dentist may be required to see a patient in person in order to request x-rays or other tests that may be required to treat (provide service) to the client 21A. In an embodiment only initial service may be completed and noted by 39N.

In an embodiment, the SPCI server 50 may provide a list of possible service providers 31 that may provide the additional service in person to a client 21A via SPCI-app on their device 20A. The list may include information about each service provider 31A including their experience, rating(s) by other clients, area of expertise, costs of service, location, and schedule availability. A client 21A via SPCI-app on their device 20A may be able to prioritize different factors about a provider 31A in order to sort the list in an embodiment, Once selected, the SPCI server 50 may forward the appointment request to the selected service provider(s) 31 via SPCI-app on their device 30A.

The selected service provider(s) may receive client 21A information via SPCI-app on their device 30A to enable them to see service performed already by other provider(s) 31B-E and other service completed and corresponding results (laboratory tests, prescriptions filled, x-rays images). The information may be provided by a CBCS 40A indirectly or directly to service provider(s) via SPCI-app on their device 30A. Such client 21A information may not be provided until the client completes various permission(s) via their client device 20A via SPCI-app on their device 20A in an embodiment. Similarly, the newly selected or assigned service provider 31A via SPCI-app on their device 30A may be able to communicate with previous service providers 31B-E for the client 21A via SPCI-app on their devices 30B-E (subject to permissions granted by the client 21A via SPCI-app on their device 20A in an embodiment).

As noted, the newly selected or assigned service provider 31A via SPCI-app on their device 30A may be able to communicate with the client 21A via SPCI-app on their device 20A. The newly selected or assigned service provider 31A via SPCI-app on their device 30A may be able to provide other service for with the client 21A via SPCI-app on their device 20A including providing treatments, recommendations, and reviews of the client's request (or case). Staff of the newly selected or assigned service provider 31A or the provider 31A may provide paperwork required for the next service or step of service electronically via SPCI-app on their device 30A or in person. In addition, Staff of the newly selected or assigned service provider 31A or the provider 31A may collect insurance or other payment for the next service or step of service electronically via SPCI-app on their device 30A or in person. The insurance coverage and payment may be performed by the SPCI architecture 10 (via the system 80A, 80B). A service provider 31A via SPCI-app on their device 30A may be able to also assign steps of elements of a service request to other providers including their staff or related professionals. The service provider 31A or other authorized providers via SPCI-app on their device 30A may be able to assign future service appointments for remote (or virtual) or in-person directly with a client 21A via SPCI-app on their device 20A.

Similarly, a service provider 31A or other authorized provider may be able or recommend other services for remote (or virtual) or in-person via SPCI-app on their device 30A to a client 21A via SPCI-app on their device 20A. Such recommendations may be based on the client's history with the SPCI architecture 10 and may include 3^{rd} party products related to past services and related services. As noted, an admin may be able to review activity within the SPCI architecture 10 via SPCI-app on their system 60A. In an embodiment, an admin may review complaints or other issues generated by clients 21 or service providers 31 and act to arbitrate such complaints or issues via electronic communications between the parties via SPCI-app on their device 60A. Such activity may be stored in a database stored in a CBCS 40A in an embodiment.

In an embodiment, all data related to service(s) conducted for a client 21A via SPCI architecture 10, 100C, 100D, 420, 400 may be stored in a database accessible to the client 21A via SPCI-app on their device 20A or a service provider 31A via SPCI-app on their device 30A (with client's permission(s)). In an embodiment, the data may be stored in a cloud distributed ledger system 40A (which may be a blockchain) to ensure the integrity of data. Via SPCI architecture 10, 100C, 100D, 420, 400 redundant services or related procedures may be avoided enabling a client 21A to receive faster and more accurate service(s).

When a client selects to request a new service (dental emergency 42P in an embodiment) (activity 152B of algorithm 150B of FIG. 5B) via SPCI-app on their device 20A, a SPCI server 50 may analyze the clients request, past requests, and related data to triage or create a concierge process for directing the request to an appropriate service provider 31A via SPCI-app on their device 30A. In an embodiment, the SPCI server 50 via a service request processing module 78A may employ artificial intelligence, machine language, and neural logic to triage a service request. A SPCI server 50 may also communicate data with an expert system 70B to help formulate questions or triage the service request(s). The expert system 70B may be IBM Watson® or other expert systems.

As shown in FIG. 3V interface 35V a SPCI server 50 may generate initial questions 36V for review by a client 21A via SPCI-app on their device 20A to determine whether a service provider 31A of SPCI architecture 10 may be able to process or provide the requested service. For example, where the client 21A is a patient and the service provider 31A is a dentist, SPCI server 50 may first ask the client 21A via SPCI-app on their device 20A showing interface 35V initial question(s) 36V (activity 154B) and refer the client 21A to another provider for service (interface 35W of FIG. 3W) (activities 156B, 158B) via SPCI-app on their device 20A in an embodiment.

In an embodiment, the SPCI server 50 may direct a client 21A via SPCI-app on their device 20A to another service provider (referral) system for initial remote communication (such a nearby (based on client's device's location) emergency medical provider web or application based interface including starting a chat session (using various media as discussed) between the client 21A and emergency service provider via SPCI-app on their device 20A. In an embodiment, a SPCI server 50 may include voice activation services or engage a voice analysis system 70A to process requests or information provided by a client 21A via SPCI-app on their device 20A. The voice activation services may employ Amazon® Alexa or Google® voice processing. In an embodiment, the voice analysis system 70A may employ Amazon® Alexa or Google® voice processing.

Based on the initial triage, a SPCI server 50 may generate a new series of questions 36X, 37X (activity 166B) based on the client's 21 service request, past requests, and related data such as shown in interface 35X of FIG. 3X and forward them to a client's 21 via SPCI-app on their device 20A (activity 168B). SPCI server 50 may also enable a client 21A via SPCI-app on their device 20A to provide a summary 36Y of request, details of request 37Y, and multimedia attachments 38Y via interface 35Y to FIG. 3Y. For a dental emergency, for example, SPCI server 50 may direct a client 21A via SPCI-app on their device 20A to take images of their teeth from different angles until a 3-D model of the client's mouth may be formed. For example, interface 35Z of FIG. 3Z may enable a client 21A via SPCI-app on their device 20A to upload multimedia including images. SPCI server 50 may then assign the service request to a service provider 31A via SPCI-app on their device 30A.

The selection of the service provider 31A may be based on the provider's availability, the client's request and the provider's associated specialization(s), schedule (whether predetermined availability schedule or real-time indicia of availability transmitted by service providers to the SPCI server using service provider devices and an associated SPCI-app), depth of queues to be processed, and past experience with the client 21A. In addition, the SPCI server 50 may evaluate the client's level of necessity for the requested service, e.g., for a medical client the SPCI server 50 may determine the client's urgency for services to address their medical need. In addition, the SPCI server 50 may compare the client's urgency for service relative to the urgency for services of other client's 20B-D having requests already in service queue(s).

Based on these factors and others a SPCI server 50 may assign a priority to the client's request and place the request in the queue of service provider's 31 via SPCI-app on their device 30A accordingly. A SPCI server 50 may report the queue status to the client 21A via SPCI-app on their device 20A as shown in interface 35AA of FIG. 3AA (activity 174B). In an embodiment, an admin via SPCI-app on their device 60A may review pending service requests and assist a SPCI server 50 with the review and determination if the request can be at least initially processed remotely (activity 172B and 174B) or in person (activity 176B), or a combination of both.

In an embodiment, a client 21A via SPCI-app on their device 20A may be presented with a list of possible service providers to select to perform their requested service. The service provider list may include information about the provider including feedback from other clients in the system, professional qualifications, availability, cost(s) of service, and other service-related information.

FIG. 5C is an algorithm 150C according to an embodiment that may be employed by a SPCI server 50 when a client requests service via SPCI-app on their device 20A (activity 152C) (patient requests service from a dentist) in an embodiment. FIG. 5D is an algorithm 150D according to an embodiment that may be employed by a SPCI server 50 and SPCI-app on a provider device 30A.

When a client requests service via SPCI-app on their device 20A (activity 152C) it may be forwarded to a service provider (activity 174C of algorithm 150C) via SPCI-app on their device 30A in an embodiment after appropriate processing. In an embodiment, when a client 21A via SPCI-app on their device 20A requests service (initiates emergency—activity 152C), the SPCI server 50 may triage the request as noted (activity 154C). In an embodiment, other client 21A information about the client as related to the service request may be provided by expert systems 70B, and other provider systems 70A. For example, in the context of a dental service consultation with a dental professional, patient brushing and flossing habits, metrics and performance evaluation conveyed to server 50 (e.g., by a third party smart toothbrush provider system 70A, directly from a smart toothbrush device in digital communication with the user's device, or via importation into the user's SPCI-app from other systems or applications implemented by the user's mobile device), in order to provide consulting professionals with additional insight into a user's oral hygiene practices. Other metrics could be provided for other service requests, such as client physical activity as recorded by their smartwatch or mobile device in an embodiment.

As also noted, a SPCI server 50 may also forward the request to an admin via SPCI-app on their device 60A for review. A SPCI server 50 may also communicate with a 3^{rd} party payor system 80B for insurance processing, pre-billing, or verification of 3^{rd} party coverage of the requested service(s). As noted, a SPCI server 50 may generate additional questions and collect additional data (activity 164C, 166C) via communications with a SPCI-app on a client's device 20A that may be stored (activity 172C) via CBCS 40A. The SPCI server 50 may then formulate a request (activity 168C) and forward the request to service provider via SPCI-app on their device 30A as described above (174C) based on various factors and using artificial intelligence and expert systems 70B. The use a CBCS 40A for all client 21A and service provider data 31 may enable a client 21A via SPCI-app on their device 20A to view all their information (including all tests, images, notes from service providers 31) anytime. In an embodiment, service provider 31A and client 21A via SPCI-apps on their devices 30A, 20A may be able to see the status of 3^{rd} party coverage or payment status prior, during, or after service processing and confirm such claims or payments to prevent fraud and expedite 3^{rd} party coverage processing and payment processing.

As shown FIG. 5D, a service provider (dentist) may review the service request (activity 176C) via SPCI-app on their device 30A and determine whether the request may be processed in person and notify the client 21A via SPCI-app on their device 20A (activity 182C), discuss request with the client to learn more about the request (issues) via SPCI-app on their device 20A (activity 184C), or discuss the request with another specialist via other provider system 70A, provider device 30A via SPCI-app on their device 30A, or admin via SPCI-app on their device 60A (activity 186C).

Based on this activity, a service provider 31A may generate an initial bill for remote interactions via SPCI-app on their device 30A (activity 178C). Then the service provider 31A via SPCI-app on their device 30A and SPCI server 50 via AI may plan method-steps to complete the service request (provide treatment) (activity 188C) and generate further billing for determined service(s) activity 192C. The service provider 31A via SPCI-app on their device 30A and SPCI server 50 may follow up on other service requests including laboratory tests and prescribed medication(s) in an embodiment (activity 194C) and further update the client's records, which may be stored via the CBCS 40A (activity 196C). If the service request is complete (activity 198C), it may be closed or additional review-steps conducted by the service provider or another service provider 31A via SPCI-app on their device 30A. In an embodiment, once at least an initial resolution is achieved or a portion of the service request in complete, payment for services may be processed via payment from the client 21A via a payment system 80A or 34 party payor system 80B, and their respective network communications interfaces 82A and 82B (activities 197C and 199C).

In an embodiment, the databases 54, 56 may employ Greenplum (www.greenplum.com), Hadoop (hadoop.apache.org) HTTP Filer Server (HFS), PostgreSQL (www.postgresql.org) software, firebase (firebase.google.com), and other software and hardware to maintain the databases 54, 56. A SPCI server 50 may also store data on one or more cloud clusters or distributed systems. The data communication module 92A may enable a SPCI server 50 to communicate over various networks using different protocols.

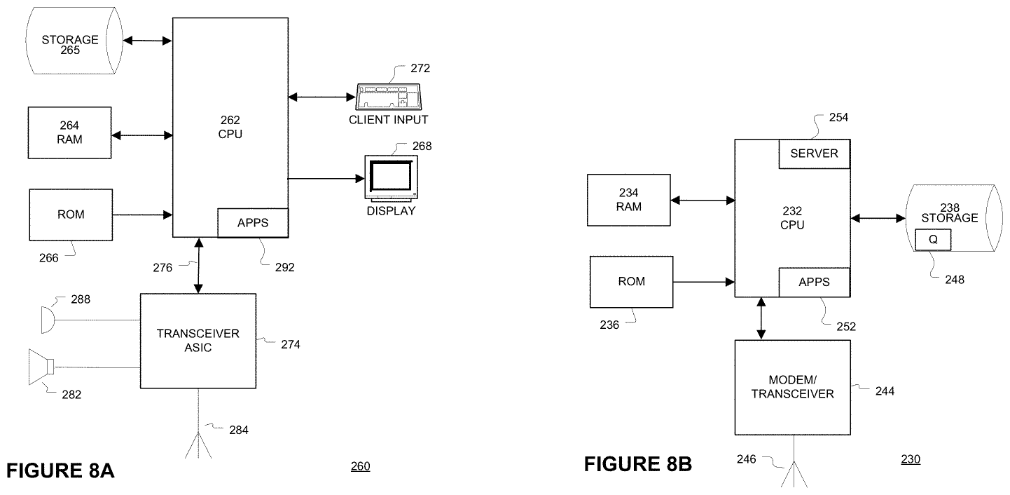

A device 260 is shown in FIG. 8A that may be used in various embodiments as a device 20A-D, 30A-B, 70A-D, 80A-B, 320A, 330A-C, and 60A-B. The device 260 may include a central processing unit (CPU) 262, a random-access memory (RAM) 264, a read only memory (ROM) 266, a display 268, a user input device 272, a transceiver application specific integrated circuit (ASIC) 274, a microphone 288, a speaker 282, a storage unit 265, and an antenna 284. The CPU 262 may include an application module 292 including an application module 292. The RAM 264 and/or storage unit 265 may store SPCI server 50A provided content, whether in a database or otherwise.

In an embodiment, the applications 292 may be a separate module. The application module 292 may process messages, displays, or pages from and generate messages, display, responses, or pages for the SPCI server 50A server 52. The storage 265 may store data provided by the SPCI server 50A server 52. The storage device 265 may comprise any convenient form of data storage and may be used to store temporary program information, queues, databases, and overhead information.

The ROM 266 may be coupled to the CPU 262 and may store the program instructions to be executed by the CPU 262, and the application module 292. The RAM 264 may be coupled to the CPU 262 and may store temporary program data, and overhead information. The user input device 272 may comprise an input device such as a keypad, touch screen, track ball or other similar input device that allows the user to navigate through menus, displays in order to operate the device 260. The display 268 may be an output device such as a CRT, LCD, touch screen, or other similar screen display that enables the user to read, view, or hear received messages, displays, or pages from the SPCI server 50A server 52.

A microphone 288 and a speaker 282 may be incorporated into the device 260. The microphone 288 and speaker 282 may also be separated from the device 260. Received data may be transmitted to the CPU 262 via a bus 276 where the data may include messages, displays, or pages received, messages, displays, or pages to be transmitted, or protocol information. The transceiver ASIC 274 may include an instruction set necessary to communicate messages, displays, or pages in architecture 10. The ASIC 274 may be coupled to the antenna 284 to communicate wireless messages, displays, or pages within the architecture 10. When a message is received by the transceiver ASIC 274, its corresponding data may be transferred to the CPU 262 via the bus 276. The data can include wireless protocol, overhead information, and pages and displays to be processed by the device 260 in accordance with the methods described herein.

FIG. 8B illustrates a block diagram of a device 230 that may be employed as a SPCI server 50A-B or CBCS 40A-B in various embodiments. The device 230 may include a CPU 232, a RAM 234, a ROM 236, a storage unit 238, a modem/transceiver 244, and an antenna 246. The CPU 232 may include a web-server 254 and application module 252. The RAM 234 may include a queue or database where the database may be used to store information including tenant, space provider, or space information, related data, and statistics. The storage 238 may also include a queue or database 248 where the database 248 may be used to store tenant, space provider, or space information. In an embodiment, the server 254 and the application module 252 may be separate elements. In an embodiment, the server 254 may generate content for web-pages or displays to be forwarded to a client device 20A.

The modem/transceiver 244 may couple, in a well-known manner, the device 230 to the network 16 to enable communication with a device 20A-B, 30A-B, and 60A-B. In an embodiment, the modem/transceiver 244 may be a wireless modem or other communication device that may enable communication with a device 20A-B, 30A-B, and 60A-B. The CPU 232 via the server 254 may direct communication between modem 244 and a client device 20A or other devices 30A, 40A, 50A, 60A, 70A, 80.

The ROM 236 may store program instructions to be executed by the CPU 232, server 254, or application module 252. The RAM 234 may be used to store temporary program information, queues, databases, and overhead information. The storage device 238 may comprise any convenient form of data storage and may be used to store temporary program information, queues, databases, and overhead information.

Any of the components previously described can be implemented in a number of ways, including embodiments in software. Any of the components previously described can be implemented in a number of ways, including embodiments in software. Thus, the CPU 232, server 254, application module 252, modem/transceiver 244, antenna 246, storage 238, RAM 234, ROM 236, database 248, CPU 262, application module 292, transceiver ASIC 274, antenna 284, microphone 288, speaker 282, ROM 266, RAM 264, user input 272, display 268, SPCI server 50A, CBCS 40A, 20A-D, 30A-B, 70A-D, 80A-B, 320A, 330A-C, and 60A-B, may all be characterized as “modules” herein.

The modules may include hardware circuitry, single or multi-processor circuits, memory circuits, software program modules and objects, firmware, and combinations thereof, as desired by the architect of the architecture 10 and as appropriate for particular implementations of various embodiments.

The apparatus and systems of various embodiments may be useful in applications other than a sales architecture configuration. They are not intended to serve as a complete description of all the elements and features of apparatus and systems that might make use of the structures described herein.

Applications that may include the novel apparatus and systems of various embodiments include electronic circuitry used in high-speed computers, communication and signal processing circuitry, modems, single or multi-processor modules, single or multiple embedded processors, data switches, and application-specific modules, including multilayer, multi-chip modules. Such apparatus and systems may further be included as sub-components within a variety of electronic systems, such as televisions, cellular telephones, video game consoles, personal computers (e.g., laptop computers, desktop computers, handheld computers, tablet computers, etc.), workstations, radios, video players, audio players (e.g., mp3 players), vehicles, medical devices (e.g., heart monitor, blood pressure monitor, etc.) and others. Some embodiments may include a number of methods.

It may be possible to execute the activities described herein in an order other than the order described. Various activities described with respect to the methods identified herein can be executed in repetitive, serial, or parallel fashion.

A software program may be launched from a computer-readable medium in a computer-based system to execute functions defined in the software program. Various programming languages may be employed to create software programs designed to implement and perform the methods disclosed herein. The programs may be structured in an object-orientated format using an object-oriented language such as Java or C++. Alternatively, the programs may be structured in a procedure-orientated format using a procedural language, such as assembly or C. The software components may communicate using a number of mechanisms well known to those skilled in the art, such as application program interfaces or inter-process communication techniques, including remote procedure calls. The teachings of various embodiments are not limited to any particular programming language or environment.

The accompanying drawings that form a part hereof show, by way of illustration and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In the foregoing Detailed Description, various features are grouped together in a single embodiment for the purpose of streamlining the disclosure. This method of disclosure is not to be interpreted to require more features than are expressly recited in each claim. Rather, inventive subject matter may be found in less than all features of a single disclosed embodiment. Thus, the following claims are hereby incorporated into the Detailed Description, with each claim standing on its own as a separate embodiment.

What is claimed is:

1. A method for a network-connected server to facilitate interactive communications between one or more dental service providers and one or more patients, the dental service provider using a network-connected service provide electronic device (SPED) and each patient using a network-connected patient electronic device (PED), the method comprising:
receiving a request for a remote consultation from a PED utilized by a patient, the request comprising one or more items of information descriptive of the consultation; and
determining suitability of the request for remote consultation based at least in part upon information provided by the PED in said request, in order to either (a) initiate a service request for a service provider remote session; or (b) direct the patient to seek an alternative service provider.

2. The method of claim 1, wherein the step of receiving a request for a remote consultation from a PED comprises:
querying the patient using the PED, for information concerning the remote consultation; and
transmitting information responsive to the step of querying the patient, to the server;
wherein the step of determining suitability of the request for remote consultation is further performed based at least in part upon information received in response to the step of querying the patient.

3. The method of claim 2, wherein the step of querying the patient comprises querying whether the patient has a condition resulting from trauma.

4. The method of claim 2, further comprising:
queueing the service request for connection with a service provider, wherein the service request is prioritized relative to other service requests based at least in part upon information received by the server in connection with the request for remote consultation.

5. The method of claim 1, in which the step of determining suitability of the request for remote consultation comprises: transmitting a notification to the PED directing the patient to visit an emergency medical services provider.

6. The method of claim 2, in which the step of determining suitability of the request for remote consultation comprises applying a machine learning component having inputs comprising the one or more items of information and/or the information responsive to the step of querying the patient, and having feedback comprising service provider evaluation of suitability following completion of a remote consultation session.

7. The method of claim 2, in which the step of querying the patient using the PED comprises requesting upload by the patient of a media content captured by the PED.

8. The method of claim 7, in which the step of determining suitability of the request for remote consultation comprises analyzing said media content by applying an image recognition component for automated identification of one or more predetermined conditions.

9. The method of claim 8, in which the one or more predetermined conditions comprise one or more of: presence of blood, and absence of a tooth.

10. The method of claim 4, in which the step of querying the patient using the PED comprises requesting upload by the patient of a media content captured by the PED; and wherein the step of queueing the service request comprises prioritizing the service request relative to other service requests based at least in part upon said media content.

11. The method of claim 10, wherein prioritizing the service request relative to other service requests is further based at least in part upon a duration of time since submission of the service request.

12. The method of claim 1, in which the step of determining suitability of the request for remote consultation further comprises evaluating records associated with the patient stored in a service provider electronic medical records (EMR) system.

13. The method of claim 1, in which the step of determining suitability of the request for remote consultation further comprises evaluating records associated with the patient stored in an insurer electronic medical records (EMR) system.

14. The method of claim 1, in which the step of determining suitability of the request for remote consultation further comprises evaluating records associated with the patient stored by the server in a patient profile.

15. The method of claim 2, in which the step of querying the patient using the PED, for information concerning the remote consultation, comprises applying a voice recognition component to procure patient information.

16. The method of claim 1, in which the step of receiving a request for a remote consultation comprises storing at least a subset of the one or more items of information descriptive of the consultation within a distributed ledger.

17. The method of claim 1, further comprising: prioritizing the service request for a service provider remote session based upon one or more prioritization factors, said prioritization factors comprising one or more of said items of information descriptive of the consultation.

18. The method of claim 17, in which said prioritization factors further comprise historical consultation information stored by or accessible to said server.

19. The method of claim 17, in which said prioritization factors further comprise a duration of time since said step of receiving a request for a remote consultation.

20. The method of claim 1, further comprising: assigning the service request to a subset of service providers based at least in part upon one or more of said items of information descriptive of the consultation.

21. The method of claim 20, wherein the subset of service providers is selected based at least in part upon service providers specialization.

22. The method of claim 20, wherein the subset of service providers is selected based at least in part upon indicia of availability received from said service providers.

23. The method of claim 20, wherein the subset of service providers is selected based at least in part upon records of prior interaction between the patient and one or more of said service providers.

24. The method of claim 1, further comprising initiating one or more of the following between the server and the PED: a network-based videoconferencing session, secure text messaging, and telephonic voice communication.

25. The method of claim 1, further comprising:
queueing the service request for initiation of a remote consultation with a service provider; and
during a period in which the service request is queued, initiating display of media content on the PED, the media content selected from amongst a plurality of media content options based on one or more personalization factors.

26. The method of claim 25, in which the one or more personalization factors comprise one or more of said items of information descriptive of the consultation.

27. The method of claim 26, wherein said media content comprises patient education information having content associated with the consultation.

28. The method of claim 25, in which the one or more personalization factors comprise profile information associated with the patient stored by the server.

29. The method of claim 25, in which the one or more personalization factors comprise historical requests for consultation submitted by the patient.

30. The method of claim 25, in which the media content comprises an advertisement.

31. The method of claim 1, in which the step of determining suitability of the request for remote consultation comprises directing the patient to seek an alternative service provider, the method further comprising: displaying contact information for one or more service providers to whom the patient is referred.

32. The method of claim 31, further comprising: automatically transmitting an appointment request to said one or more service providers to whom the patient is referred.

33. A remote interactive service system that enables patients to remotely request service from a service provider, including:
a plurality of patient electronic devices (PED), each PED including an interface capable of communicating one of wired and wirelessly;
a plurality of service provider electronic devices (SPED), each SPED including an interface capable of communicating one of wired and wirelessly;
a remote interactive service database maintaining transactions conducted in the system;
a remote, network-connected service provider client interactive system (SPCI), including a server for communicating with the plurality of PED and the plurality of SPED; and
the SPCI via the server:
enables one of a plurality of clients to remotely request service via one of the plurality of PED, each patient of the plurality of patients having a unique identifier in the system;
reviews past service requests of the one of a plurality of patients via their unique identifier stored in the remote interactive service database;
triages the one of a plurality of patients' service request by sending and receiving the response for at least one related service question from the one of a plurality of patients via one of the plurality of PED; and
based on the received response, one of: (a) generates a related service request to one of a plurality of service providers via one of the plurality of the SPED, and (b) directs the one of a plurality of patients to seek service via a different service provider.

34. The remote interactive service system of claim 33, wherein remote interactive service database is maintained independent of the SPCI.

35. The remote interactive service system of claim 34, wherein remote interactive service database is stored in a distributed ledger.

36. The remote interactive service system of claim 33, wherein the SPCI, via the server based on the received response, one of: (a) generates a related service request to one of a plurality of service providers based at least on the one of a plurality of patients' past transactions stored in the database based on their unique ID and the plurality of service providers past transactions stored in the database based on their unique ID via one of the plurality of SPED; and (b) directs the one of a plurality of patients to seek service via a different service provider.

37. The remote interactive service system of claim 33, wherein each service provider of the plurality of service providers via their unique ID may indicate their availability to process a service request via one of the plurality of SPED.

38. The remote interactive service system of claim 37, wherein the availability of each service provider of the plurality of service providers is stored in the remote interactive service database based on each service provider's unique ID; and wherein the related service request is further generated based on the availability of the service providers.

39. The remote interactive service system of claim 36, wherein the one of a plurality of service providers receiving the related service request is able to communicate via their SPED with the one of the plurality of patients and the one of the plurality of patients' PED.

40. The remote interactive service system of claim 39, wherein the remote interactive service database further stores all communications associated with a unique ID of one or more of the patient and the service provider.

41. The remote interactive service system of claim 33, wherein the server is further configured to electronically submit prescriptions for a patient to one or more pharmacies for fulfillment in response to submission of a prescription request from a service provider via their SPED.

42. The remote interactive service system of claim 41, wherein the electronically submitted prescriptions are stored in the remote interactive service database based on the unique ID of a submitting service provider and the unique ID of a patient for whom the prescription was submitted.
